SEQUENCE LISTING (10 sequences)

SEQ ID NO: 1 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 339 aa, Homo sapiens
AEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 776 aa, Homo sapiens [sequence omitted]

SEQ ID NO: 8 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKLPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 9 - protein, 316 aa, Homo sapiens
MLRALQQRKREAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 10 - protein, 307 aa, Homo sapiens
MGTVLSLSPSYRKATLFEDGAATVGHYTAVQNSKNAKDKNLKRHSIISVLPWKRIVAVSAKKKNSKKVQPNSSYQNNITHLNNENLKKSLSCANLSTFAQPPPAQPPAPPASQLSGSQTGGSSSVKKAPHPAVTSAGTPKRVIVQASTSELLRCLGEFLCRRCYRLKHLSPTDPVLWLRSVDRSLLLQGWQDQGFITPANVVFLYMLCRDVISSEVGSDHELQAVLLTCLYLSYSYMGNEISYPLKPFLVESCKEAFWDRCLSVINLMSSKMLQINADPHYFTQVFSDLKNESGQEDKKRLLLGLDR

(19) United States
(12) Patent Application Publication
(10) Pub. No.: US 2011/0263696 A1
(43) Pub. Date: Oct. 27, 2011

(54) PROANTHOCYANIDINS FROM CINNAMON AND ITS WATER SOLUBLE EXTRACT INHIBIT TAU AGGREGATION

(76) Inventors: Donald J. Graves, Santa Barbara, CA (US); John Lew, Goleta, CA (US)

(21) Appl. No.: 12/593,800

(22) PCT Filed: Mar. 31, 2008
(86) PCT No.: PCT/US08/04236

§ 371 Date: Sep. 29, 2009

Related U.S. Application Data
(60) Provisional application No. 60/921,204, filed on Mar. 30, 2007.

(51) Int. Cl. A61K 31/353; C07D 311/04; C07C 45/78; A61P 25/00; C07D 407/10; A61K 31/11; A61P 25/28; C07K 1/00; C07C 47/21

(52) U.S. Cl. 514/456; 530/410; 549/399; 568/438; 568/425; 514/701

(57) ABSTRACT

Compositions comprising proanthocyanidin compositions (e.g. those extracted from cinnamomum species) that are observed to bind tau and inhibit its aggregation as well as methods for making and using such compositions are disclosed. In certain embodiments of the invention, the proanthocyanidins can be used as a probe to identify and/or characterize tau isoforms in a variety of contexts. In other embodiments of the invention, these compositions are used in methods designed to treat neurological disorders associated with tau aggregation (e.g. Alzheimer's disease).

### CROSS REFERENCE TO RELATED APPLICATIONS

This application claims the benefit of U.S. provisional patent application No. 60/921,204, filed Mar. 30, 2007, the entire contents of which are incorporated herein by reference.

### STATEMENT REGARDING FEDERALLY SPONSORED RESEARCH AND DEVELOPMENT

The present invention was made with Government support under Grant No. GM058445 awarded from the National Institutes of Health. The Government has certain rights in this invention.

### FIELD OF THE INVENTION

The present invention relates to cinnamon proanthocyanidin extracts and/or proanthocyanidin compositions, as well as methods for using such extracts and/or compositions to bind tau, a polypeptide observed to exhibit abnormal aggregation in neurodegenerative pathologies such as Alzheimer's disease.

### BACKGROUND OF THE INVENTION

Incorrect folding or mis-folding of proteins and the formation of aggregates is associated with various diseases including diabetes, Parkinson's disease, and Alzheimer's disease. Analysis of protein deposits in the brains of individuals with Alzheimer's disease shows the formation of amyloid plaques and neurofibrillary tangles associated with neurons. While it is not certain what structures of proteins are cytotoxic to neurons, the identified filaments or soluble protein aggregates are believed to play a role involved in pathologies such as Alzheimer's disease because the appearance of these lesions largely correlates with pathological neurofibrillary degeneration and brain atrophy, as well as with cognitive impairment.

Both amyloid plaques and neurofibrillary tangles contain paired helical filaments (PHFs), of which a major constituent is the microtubule-associated protein tau. Plaques also contain extracellular β-amyloid fibrils derived from the abnormal processing of amyloid precursor protein. Studies of Alzheimer's disease indicate that the loss of the normal form of tau, accumulation of pathological PHFs and loss of synapses in the mid-frontal cortex correlate with associated cognitive impairment. Furthermore, loss of synapses and loss of pyramidal cells both correlate with morphometric measures of tau-reactive neurofibrillary pathology, which parallels, at a molecular level, an almost total redistribution of the tau protein pool from a soluble to a polymerized form (PHFs) in Alzheimer's disease.

Tau polypeptide exists in alternatively-spliced isoforms, which contain three or four copies of a repeat sequence corresponding to the microtubule-binding domain. Tau in PHFs is proteolytically processed to a core domain involved in tau-tau interactions. Once formed, PHF-like tau aggregates act as seeds for the further capture and provide a template for proteolytic processing of full-length tau protein. In the course of their formation and accumulation, paired helical filaments (PHFs) assemble to form amorphous aggregates within the cytoplasm, probably from early tau oligomers which become truncated prior to, or in the course of, PHF assembly. These filaments then go on to form classical intracellular neurofibrillary tangles. The neurofibrillary tangle assembly process consumes the cellular pool of normal functional tau and inducing new tau synthesis. Eventually, functional impairment of the neuron progresses to the point of cell death, leaving behind an extracellular tangle.

Tau within PHFs appears to undergo conformational changes during incorporation into the filaments. During the onset of Alzheimer's disease, this conformational change could be initiated by the binding of tau to a pathological substrate, such as damaged or mutated proteins. In Alzheimer's disease, typical pharmaceutical therapies focus on symptomatic treatment of the loss of cholinergic transmission which results from neurodegeneration. However, although currently available therapeutic regimens can delay progression of the disease, they do not prevent or reverse it. The identification of compositions that inhibit or even reverse the aggregation of tau therefore provides another strategy for prophylaxis and/or for inhibiting the progression of the disease.

Although reagents and assays for the examination of tau aggregation are known in the art, the identification of further reagents that for example, can be used to bind tau and modulate its aggregation is desirable. Such reagents and associated assays can be used for example to identify and characterize inhibitors and/or modulators of tau-tau association and tau-tau aggregation. In addition, reagents that bind tau and modulate its aggregation can be used in a variety of diagnostic, prognostic or therapeutic methodologies that are used to identify, characterize and treat conditions such as Alzheimer's disease.

### SUMMARY OF THE INVENTION

Embodiments of the present invention relates to the disclosure provided herein that proanthocyanidins (e.g. those derived from cinnamon extracts) can bind to tau polypeptides, can inhibit polypeptide aggregation and can protect soluble tau polypeptides from aggregation. In this context, embodiments of the invention include proanthocyanidin compositions as well as methods for making and using such compositions. In an illustrative embodiment of the invention, the disclosure provides methods for using isolated proanthocyanidins to bind tau isoforms. Optionally these methods further inhibit or reduce the formation of tau aggregates, a phenomenon observed in neurodegenerative diseases such as Alzheimer's disease.

The invention disclosed herein has a number of embodiments. One embodiment of the invention is a method of binding a mammalian tau polypeptide with an isolated proanthocyanidin by combining the tau polypeptide with an isolated proanthocyanidin composition and then allowing an isolated proanthocyanidin in the composition to bind the tau polypeptide so that the tau polypeptide is bound by the isolated proanthocyanidin. In typical embodiments of this method, proanthocyanidin(s) bound to the tau polypeptide can be used to observe the presence of tau polypeptides in a biological sample (e.g. an aggregation of tau polypeptides in a biological sample). These binding methods can be used in a variety of contexts. For example, embodiments of these methods can include using observations of the presence of an aggregation of tau polypeptides to diagnose a tauopathy (e.g. Alzheimer's disease).

In certain embodiments of the invention, binding of tau polypeptide by an isolated proanthocyanidin can be used to perturb the ability of the tau polypeptide to form an aggregation of tau polypeptides. Optionally, such methods can be performed in vivo in a mammal having a neurological condition or disorder. In an illustrative example of this, the binding of the tau polypeptide by the isolated proanthocyanidin can be used to perturb the ability of tau polypeptides to form an aggregation in an individual suffering from a tauopathy. One illustrative embodiment of the invention is a method of treating a mammal having a neurological condition or disorder characterized by an aggregation of tau polypeptides (e.g. Alzheimer's disease), comprising administering to said mammal an effective amount of an isolated proanthocyanidin composition selected for its ability to perturb aggregation of tau polypeptides. Optionally in these methods, the isolated proanthocyanidin composition comprises at least one of an A-linked proanthocyanidin dimer, trimer, tetramer, or pentamer; a proanthocyanidin B2; a cinnamaldehyde; an oxidized catechin; or an oxidized epicatechin.

Yet another embodiment of the invention is a method determining if a proanthocyanidin compound binds to a tau polypeptide comprising combining the tau polypeptide with a proanthocyanidin compound and then testing the combination of the tau polypeptide and the proanthocyanidin compound to determine if the proanthocyanidin compound binds the tau polypeptide. A related embodiment of the invention is a method of determining if an isolated proanthocyanidin compound perturbs aggregation of tau polypeptides comprising observing the ability of tau polypeptides to aggregate in the absence of the isolated proanthocyanidin compound; combining tau polypeptides with the isolated proanthocyanidin compound and observing the ability of tau polypeptides to aggregate in the presence of the isolated proanthocyanidin compound, wherein a decrease in tau aggregation observed in the presence of the isolated proanthocyanidin compound as compared to the amount of tau aggregation observed in the absence of the isolated proanthocyanidin compound identifies the isolated proanthocyanidin compound as perturbing the ability of tau polypeptides to aggregate.

As discussed in detail below, the proanthocyanidin compositions used in embodiments can be obtained from a variety of sources. Typically however, the isolated proanthocyanidin composition is derived from Cinnamomum loureirii, Cinnamomum cassia or Cinnamomum zyelanicum. Optionally, the isolated proanthocyanidin composition is derived from an aqueous extract of these Cinnamomum species and comprises at least one of the following compounds: an A-linked proanthocyanidin dimer, trimer, tetramer, or pentamer; a proanthocyanidin B2; a cinnamaldehyde; an oxidized catechin; or an oxidized epicatechin. In certain embodiments of the invention, the proanthocyanidin composition comprises at least the A-linked proanthocyanidin trimer Another embodiment of a proanthocyanidin composition of the invention comprises isolated proanthocyanidin compound derived from Cinnamomum loureirii, Cinnamomum cassia or Cinnamomum zyelanicum and characterized by at least one of the following properties: an ability to bind tau polypeptides; an ability to reduce the ability of tau polypeptides to form an aggregation as observed by a thioflavin T assays; an ability to reduce the number and length of tau filament formation as measured by electron microscopy studies, exhibits a decrease in the absorbance spectra upon combination with tau polypeptides; or comprises: a B-linked proanthocyanidin dimer having a mass spectroscopy peak of 617 m/z; an A-linked proanthocyanidin trimer having a mass spectroscopy peak of 905 m/z; an A-linked proanthocyanidin tetramer a mass spectroscopy peak of 1193 m/z; or an A-linked proanthocyanidin pentamer having a mass spectroscopy peak of 1481 m/z; a proanthocyanidin B2; a cinnamaldehyde; an oxidized catechin; or an oxidized epicatechin.

A discussed in detail below, embodiments of the invention include methods for making the proanthocyanidin compositions disclosed herein. One illustrative embodiment of this is a process for preparing an isolated proanthocyanidin composition comprising: extracting a sample of Cinnamomum loureirii, Cinnamomum cassia or Cinnamomum zyelanicum with an aqueous solution; agitating this extracts for at least one minute at a temperature between 40-90° C.; centrifuging the agitated extract so as to produce a first pellet and a first supernatant; incubating the first supernatant of at 0-4° C. for at least one minute; centrifuging the incubated supernatant so as to produce a second pellet and a second supernatant; and then filtering the second supernatant, wherein the resultant filtered second supernatant comprises an aqueous solution of the isolated proanthocyanidin composition. Optionally the method further comprises lyophilizing the aqueous solution of the isolated proanthocyanidin composition so as to form a solid isolated proanthocyanidin composition. Embodiments of the invention further include an isolated proanthocyanidin composition product produced by such processes.

As noted above, embodiments of the invention include proanthocyanidin compositions, as well as methods for using such compositions to bind tau isoforms and/or inhibit or reduce the formation of tau aggregates, a phenomenon observed in neurodegenerative diseases such as Alzheimer's disease. In this context, embodiments of the invention also include articles of manufacture and/or kits designed to facilitate the diagnostic and/or therapeutic methods of the invention. Typically, such kits include instructions for using the elements therein according to the methods of the present invention. Such kits can comprise a carrier means being compartmentalized to receive in close confinement one or more container means such as vials, tubes, and the like, each of the container means comprising one of the separate elements to be used in the method. For example, one of the containers can comprise tau and/or specific proanthocyanidin compounds (e.g. A-linked proanthocyanidin trimer), and/or cinnamon proanthocyanidin extracts for example. In a typical embodiment of the invention, an article of manufacture containing materials useful for the examination and/or treatment of the disorders described herein is provided. The article of manufacture comprises a container and a label. Suitable containers include, for example, bottles, vials, syringes, and test tubes. The containers may be formed from a variety of materials such as glass or plastic. The container can hold a composition (e.g. a cinnamon proanthocyanidin extracts and/or proanthocyanidin compositions) that is effective for examining or modulating tau in mammals. The label on, or associated with, the container indicates that the composition is used for examining and/or modulating the conformation of tau isoforms. The article of manufacture may further comprise a second container comprising a buffer, such as phosphate-buffered saline, Ringer's solution and dextrose solution. It may further include other materials desirable from a commercial and user standpoint, including other buffers, diluents, filters, needles, syringes, and package inserts with instructions for use.

### BRIEF DESCRIPTION OF THE FIGURES

FIG. 1. A) ESI mass spectrum of proanthocyanidins in filtered extract from C. zeylanicum observed as (M+K⁺) ions. B-type dimer, A-type trimer, tetramer and pentamer are observed B). Structure of Proanthocyanidins. Proanthocyanidins from cinnamon are primarily oligomers of epicatechin in which B-type oligomers are generated by successive linkage through the C4-A8 carbon atoms. Two electron oxidation of the B-type dimer gives the corresponding A-type dimer. (Figure taken from Dixon, R A et al. New Phytol. (2005) 165:9-28).

FIG. 2. UV absorbance spectra of purified A-type proanthocyanidin trimer as a function of increasing tau concentration. The curves from top to bottom (at A280) are as follows: 0 uM; 4.4 uM; 8.8 uM; 17.6 uM tau¹⁸⁷; proanthocyanidin trimer=15 uM. As is known in the art, the isobestic points are found where the curves overlap.

FIG. 3. Proanthocyanidins inhibit tau aggregation. A) Turbidity assay. Aggregation of FL-tau (50 uM) was initiated with heparin (0.1 mg/ml) in 20 mM NaPO4, pH 7.0. Curve 1: no proanthocyanidin; Curve 2: 200 uM purified A-type trimer. B) After 45 min, reactions were centrifuged at 100,000 g×60 min. The amount of tau in the pellets (P) vs. supernatants (S) were examined by SDS-PAGE. Markers (top to bottom) are 97, 66, 45 kDa. C, D) EM after aggregation for 45 min. Scale bars=500 nm; C) no proanthocyanidin, D) 200 uM purified A-type trimer.

FIG. 4. Cinnamon extract inhibits tau aggregation. Full-length tau (50 uM) was induced to undergo aggregation with heparin (0.1 mg/ml) for 16 hr at room temp in the presence of varying concentrations of CE. Samples were then treated with 1% sarkosyl for 1 hr, centrifuged (105,000 g×1 hr), and the pellets (P) versus supernatants (S) then analyzed by SDS-PAGE. Increasing CE concentration results in less tau in the pellet fractions. Arrow: tau; M: markers—66 and 45 kDa, from top to bottom.

FIG. 5. Full-length tau (FL-tau) versus tau¹⁸⁷. The four repeat sequences at the C-terminus constitute the microtubule binding domain. Inter-repeat sequences 1 and 2 each contain one of two hexapeptide motifs essential for aggregation of tau into fibers. The two repeat sequences at the N-terminus in FL-tau have unknown function.

FIG. 6. Proanthocyanidins inhibit aggregation of tau¹⁸⁷. Aggregation of tau¹⁸⁷ (50 uM) was initiated with heparin (0.1 mg/ml) in 20 mM NaPO₄, pH 7.0. in the presence of: no proanthocyanidin (left panel), or 200 uM purified A-type trimer (right panel). Scale: Bars=500 nm

FIG. 7. Cinnamon extract does not compete with heparin for binding to tau. Tau¹⁸⁷ was incubated with heparin-Sepharose in the presence of increasing concentrations of cinnamon extract for 5 min, then centrifuged. Even at 5× the concentration used for aggregation (0.5 mg/ml final), CE did not prevent binding of tau¹⁸⁷ to heparin beads. P-pellet, S-supernatant, M-markers: 31 and 21 kDa from top to bottom.

FIG. 8. Cinnamon extract disassembles pre-existing tau fibers. Tau¹⁸⁷ (50 uM) was induced to undergo aggregation with heparin (0.1 mg/ml), and fibers were visualized after 24 hrs (left panel) incubation at room temp. The sample was then divided in half, water was added to one-half (middle panel) while cinnamon extract (0.11 mg/ml) was added to the other (right panel). Fibers were visualized again after another 24 hrs. Scale: Bar=500 nm.

FIG. 9. Cinnamon extract does not inhibit tau-dependent MT assembly. Tubulin (5 uM) was incubated (a) alone, or with (b) CE, (c) tau or (d) both. MT assembly was monitored by turbidity at OD₃₅₀ in a continuous spectrophotometric assay.

FIG. 10. Cinnamon extract inhibits cdk5/p25 in vitro. Phosphorylation of histone H1 (100 uM) (ATP=100 uM) by purified cdk5/p25 was followed in a radio labeled assay in the presence of varying concentrations of cinnamon extract. Curve fitting to the following model: y=−ax/(b+x)+c reveals an IC₅₀=3.6 ug/ml extract.

FIG. 11. Dose response of cinnamon (oxidized) on tau aggregation. The data in this graph shows a dose response of cinnamon on tau aggregation via light scattering experiments. In this experiment, cinnamon was incubated overnight in 10 mM NH4OH.

FIG. 12. Effect of cinnamon A trimer on light scattering of tau. The data in this graph provides evidence that oxidized cinnamon is more effective in causing a decrease in light scattering as measured by the increase in absorbance at 350 nm than non-oxidized cinnamon A.

FIG. 13. Effect of oxidized cinnamon on tau filament formation. The data in these panels shows the inhibition of tau aggregation probed by Electron Microscopy (50 μM tau 187his+0.4 mg/ml heparin). Left panel: no cinnamon; right panel 10 μM oxidized cinnamon.

FIG. 14A. Co-expression of CDK5 ad p25 in COS-7 cells is toxic. Toxicity is seen as shrunken cell nuclei visualized by DAPI staining (red arrows). The corresponding cells express CDK5 (green fluorescent protein) and p25 (Cy3 red). Cells that do not express CDK5 or p25 have normal nuclei (DAPI). FIG. 14B. Cinnamon extract prevents Cdk5/p25—induced toxicity. Cos-7 cells expressing CDK5 and p25 (red arrows) were treated with 0.05 mg/ml of cinnamon extract. Under these conditions, toxicity due to CDK5/p25 co-expression was not observed.

FIG. 15A. Inhibition of tau aggregation by cinnamaldehyde. 50 μM Tau¹⁸⁷ was incubated with 0.11 mg/ml heparin (18 kDa) at 23° C. Aggregation was monitored by turbidity (OD350). Cinnamaldehyde was present at a final concentration of 0, 50 and 100 μM. FIG. 15B. Electron microscopy of tau fibers in the presence of cinnamaldehyde (CA). Tau fibers were visualized by electron microscopy after 5 hrs of aggregation in the presence of: 0 CA (left panel); B) 100 uM CA (right panel). Fiber density is dramatically reduced by cinnamaldehyde. FIG. 15 C. Trans-cinnamaldehyde chemical structure.

FIG. 16A. Oxidation of epicatechin monomer results in tau aggregation inhibitory activity in vitro. 50 uM tau187 was incubated with 0.11 mg/mL heparin (18 kDa) at 23° C. Epicatechin was untreated, treated with 10 mM NH4OH for 10 min at 95° C. then neutralized, or treated with 100 U of mushroom tyrosinase (Sigma) for 10 min. Epicatechin was added to the reaction at a final concentration of 25 uM. Fiber formation was monitored by OD 350. FIG. 16B. epicatechin and catechin chemical structures.

FIG. 17A. HPLC analysis of proanthocyanidin A-type trimer, A) purified or B) in whole cinnamon extract. The trimer corresponds to the major peak at ˜40 min. Areas underneath the peaks in panels A and B each correspond to 6150 pmoles. Equivalent amounts of purified trimer and trimer in cinnamon extract were tested in tau aggregation assays (see FIG. 17B). FIG. 17C. Whole cinnamon extract inhibits tau aggregation more potently than does proanthocyanidin A-type trimer alone. 50 uM tau187 was incubated with 0.11 mg/mL heparin (18 kDa) at room temperature. Fiber formation was monitored by OD 350. Concentrations of A-trimer in pure form and in whole cinnamon extract (CE) were normalized by absorbance measurements of the A-trimer resolved by HPLC. The final concentration of the trimer in the aggregation assay was 12.5 uM, added either as pure trimer or as whole extract. The corresponding concentration of the whole cinnamon extract was 0.22 mg/mL.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

Before the present cinnamon proanthocyanidin extracts and/or proanthocyanidin compositions, methods and assays are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims. All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

It must be noted that as used herein and in the appended claims, the singular forms “a”, “and”, and “the” include plural referents unless the context clearly dictates otherwise. Thus, for example, reference to “a proanthocyanidin” includes a plurality of such proanthocyanidins and reference to “a probe” includes reference to one or more probes and equivalents thereof known to those skilled in the art, and so forth. All numbers recited in the specification and associated claims (e.g. a mass spectroscopy peak of 905 m/z; 40° C. water etc.) are understood to be modified by the term “about”.

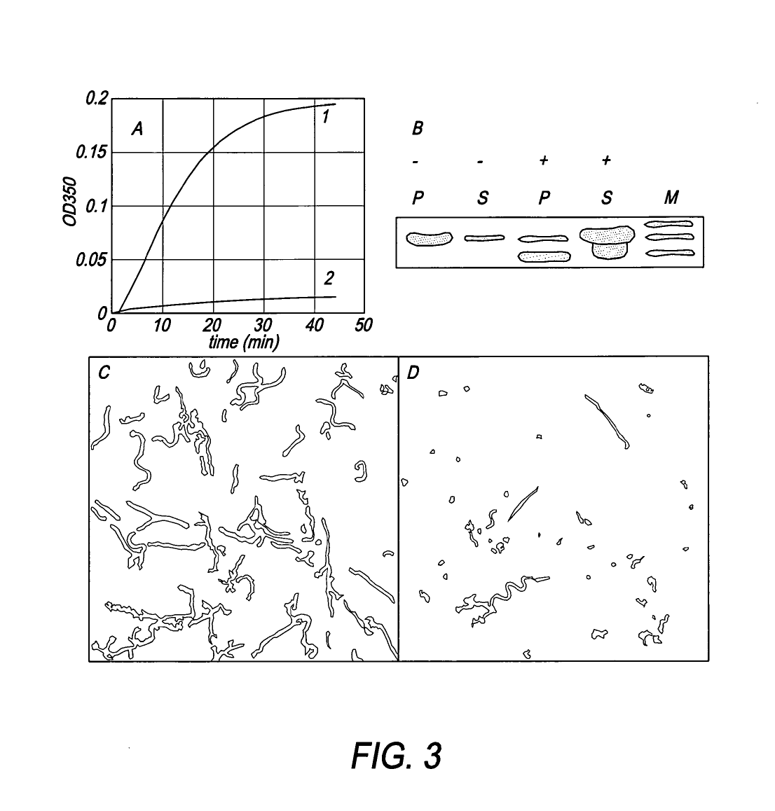

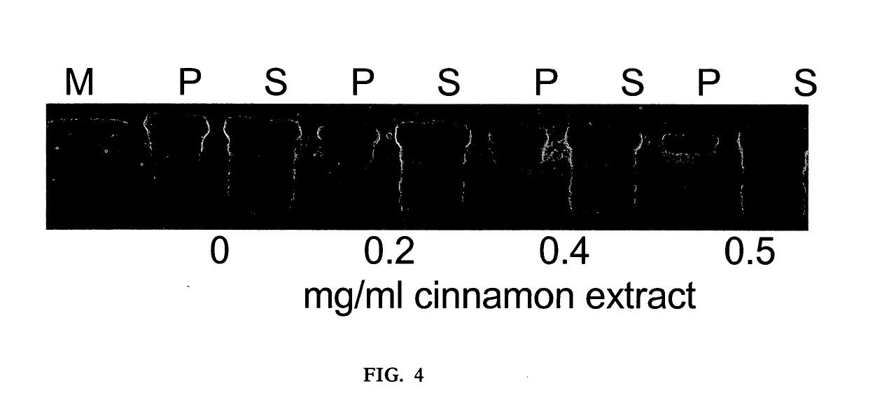

“Cinnamon proanthocyanidin extracts” as used herein refers to extracts of cinnamomum species (typically water extracts) that comprise tau binding proanthocyanidins such as proanthocyanidin type A trimers, and related bio-active proanthocyanidins (as can be readily shown by protocols such as those used to obtain the data shown in FIGS. 3 and 4). Typically, such extracts comprise water extracts of cinnamomum loureirii, cinnamomum cassia or cinnamomum zyelanicum and contain compounds such as cinnamon A, cinnamon A multimers (e.g. dimers, trimers etc.), oxidized cinnamon, non-oxidized cinnamon, cinnamon trimer, proanthocyanidin A, proanthocyanidin B2, cinnamon proanthocyanidin A, cinnamaldehydes, polyphenols such as polyphenol type-A polymers or other cinnamon extract compounds known in the art to potentiate insulin signaling. In this context, it is known that individuals with diabetes are more prone to get Alzheimer's disease since changes in tau are seen in animals defective in insulin signaling. Cinnamon extracts as used herein can comprise polyphenol type-A or type B oligomers from cinnamon with insulin-like biological activity. The cinnamon extracts of the present invention can be provided in a lyophilized form, a water extract, an extract of any other solvent, in a reconstituted form or the like. For example, the reconstituted extracts can comprise lyophilized extract dissolved in an aqueous solution. Additionally, the various compounds described in the publications cited herein (e.g. U.S. Pat. No. 6,200,569) are contemplated components of compositional embodiments of the invention that can be used with the methods of the invention.

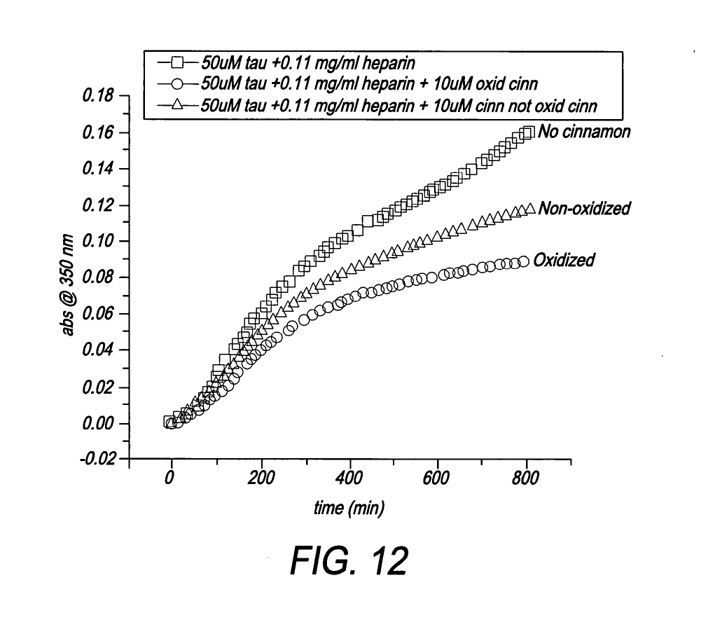

“Proanthocyanidin” is used herein according to its art accepted meaning and includes for example proanthocyanidin type A or B oligomers, proanthocyanidin polymers, and proanthocyanidin isomers, including optical isomers and mixtures thereof, as well as combinations of proanthocyanidins such as combinations of proanthocyanidin monomers, dimers, trimers, tetramers, or pentamers etc. and mixtures thereof as occur in certain embodiments of the invention (e.g. cinnamon proanthocyanidin compositions produced by the processed disclosed herein). As used herein, “cinnamon proanthocyanidins” refers to those proanthocyanidins derived from cinnamomum species such as cinnamon loureirii, and/or cinnamomum cassia and/or cinnamomum zyelanicum (e.g. proanthocyanidins derived from water extracts of these cinnamon species). In certain embodiments of the invention, the oxidation state of a component of a proanthocyanidin and/or the associated constituents in the cinnamon extracts disclosed herein can be manipulated (e.g. oxidized or reduced) as part of the preparation process, for example by exposing them to one of the variety of oxidizing or reducing agents known in the art. In this context, the proanthocyanidins and/or the associated constituents in the cinnamon extracts can be provided in a variety of forms known in the art, for example in oxidized or non-oxidized forms (see, e.g. FIG. 12 and Pavlovich et al., (2005), “Electrospray MS Profiling of Proanthocyanidin Oligomers in Commercially Available Varieties of Cinnamon and Cassia”, 53rd ASMS Conference on Mass Spectrometry and Allied Topics, San Antonio, Tex., June 5-9, ThP 318; and Lampke et al., Activation of Insulin-like Activity of Proanthocyanidins from Cinnamon”, FASEB Meeting, San Francisco, Calif., Abs. 612.2). In addition, these can be prepared according a variety of art accepted methods, for example as a pharmaceutical composition comprising a proanthocyanidin extract and a pharmaceutically acceptable carrier, or contained within a pharmaceutical dosage such as a capsule or tablet or the like.

“Isolated,” when used to describe the various proanthocyanidin extracts and/or proanthocyanidin compositions and compounds disclosed herein, means a proanthocyanidin that has been separated and/or recovered from a component of its natural environment. Contaminant components of its natural environment are materials that would typically interfere with diagnostic or therapeutic uses for the compound, and may include various compounds including proteinaceous or non-proteinaceous solutes. Ordinarily, an isolated proanthocyanidin compounds will be prepared by at least one purification step. In certain embodiments, an isolated proanthocyanidin will be prepared by at least: (1) a water extraction step such as is described in the examples below, or (2) purification to homogeneity; or (3) purification in situ within cells, since at least one component of its natural environment will not be present.

“Tau” as referred to herein comprises those tau polypeptide sequences that are observed to occur in nature or derivatives thereof (e.g. tau187) and are bound by a proanthocyanidin compound, for example the various post-transcriptionally and post-translationally processed species of the tau gene. The tau protein is a microtubule-associated phosphoprotein that stabilizes the cytoskeleton and contributes to determining neuronal shape. Tau can have an apparent molecular weight of about 55 kDa. In a normal brain, the tau protein and tau fragments typically exist in an unphosphorylated, or dephosphorylated state. However, in neurofibrillary tangles, both tau protein and tau fragment can be found in an abnormally phosphorylated state, a hyperphosphorylated state. Hyperphosphorylation impairs tau protein's ability to interact with microtubules (see, e.g., U.S. Pat. No. 6,803,233, the contents of which are herein incorporated by reference). In the human CNS, tau protein exists as at least six predominant isoforms that result from alternative splicing of a single transcript derived from a gene located on chromosome 17 (see, e.g. Goedert et al., Neuron 3, 519-526 (1989). At least three isoforms contain three microtubule binding repeats (3R), and a least three isoforms contain four (4R). The isoforms are further differentiated from each other by the presence of 0, 1, or 2 exons in the amino third of the molecule (e2 and e3). The resultant isoforms of tau form the abnormal polymeric straight (SF) and paired helical (PHF) filaments that compose the neurofibrillary tangles (NFTs), neuropil threads, and dystrophic neurites surrounding the senile plaques in Alzheimer's disease (AD) brain. Deposition of filamentous tau has been implicated in other neurodegenerative diseases in addition to AD. For example, cortical basal degeneration (CBD), progressive supranuclear palsy (PSP), Pick's disease, certain forms of Parkinson's disease, and many frontal lobar atrophies contain degenerating neurons and/or glia with polymeric tau inclusions (Feany et al., Ann. Neurol. 40, 139-148 (1996). The ultrastructural character, distribution, and biochemistry of tau inclusions differ among the various aggregate/tangle-forming diseases, now termed “tauopathies” (see, e.g. Spillantini et al., P.N.A.S. USA 94, 41130-4118 (1997). For example, the NFTs found in PSP are composed primarily of 15- to 18-nm SFs derived from a tau doublet (68 and 64 kDa) of 4R isoforms, whereas those found in Pick bodies are wide PHFs (160-nm half-period, 24-nm width) of 3R isoforms. In addition, the PHF-like deposits found in CBD demonstrate a maximum width of 27 nm and a significantly longer periodicity of 200 nm (see, e.g. Ksiezak-Reding et al., Am. J. Pathol. 145, 1496-1508 (1994)). A multiple-system tauopathy with presenile dementia (MSTD) has been described which presents with twisted filaments of 90- to 130-nm periodicity composed exclusively of four repeat tau isoforms. In addition, inherited mutations of the tau gene have been discovered in cases of frontal lobar atrophies termed frontal temporal dementia and parkinsonism (FTDP-17) (see, e.g. Hutton et al., Nature 393 702-705 (1998); Poorkaj et al., Ann. Neurol. 43, 815-825 (1998); Spillantini et al., P.N.A.S. USA 95, 7737-7741 (1998); and King et al., J. Neurochem., 74, 1749-1757 (2000)). “Tauopathy Inhibitors” as used herein refers to compositions that can inhibit tauopathies and can comprise for example cinnamon extracts, proanthocyanidin compositions, pharmaceutical compositions of these components or the like. Such inhibitors can inhibit for example tauopathies such as tau aggregation, tau phosphorylation, fibril formation, amyloid plaques, neurofibrillary tangles, paired helical filaments, filaments, or the like.

“Aggregate” is used according to its art accepted meaning and comprises for example the association, accumulation or fibrillization of tau isoforms. As is known in the art for example, tau can aggregate in certain disease states with other proteins and/or with itself. These accumulations can be fibrillizations for example. These fibrillizations can be paired helical formations (PHF). Tau can either be phosphorylated, hyperphosphorlyated, and/or unphosphorylated when forming these associations. Tau can also be in its native state, fragmented state, and/or in a mutated state in these aggregations. These aggregations can comprise misfolded or incorrectly folded tau proteins.

“Neurofibrillary tangles” are intraneuronal accumulations of filamentous material in the form of loops, coils or tangled masses. They are typically present in Alzheimer's disease patients in parts of the brain associated with memory functions, such as the hyppocampus and adjacent parts of the temporal lobe. Neurofibrillary tangles can also be found during normal aging of the brain, however, they are found in a significantly higher density in the brain of Alzheimer's disease patients, and in the brains of patients with other neurodegenerative diseases, such as progressive supranuclear palsy, postencephaltic Parkinson disease, Pick's disease, amylotrophic lateral sclerosis, etc. Previous studies suggest that, among other things, neurofibrillary tangles can significantly contribute to the cognitive decline associated with the disease and also directly to neuronal cell death. Ultrastructurally, neurofibrillary tangles are composed predominantly of paired helical filaments. A major component of PHF is an abnormally phosphorylated form of a tau and its fragments.

The term “biological sample” is used herein according to its broadest meaning and refers to the wide variety of biological materials that can be analyzed in the methods of the present invention. Biological materials typically analyzed in such methods include tissues from brain. Such materials include in vivo and in vitro cellular materials such as materials from biopsies and/or materials from in vitro cell lines as well as compositions comprising mammalian macromolecules. The macromolecules analyzed in these materials typically include polypeptides such as the various tau isoforms known in the art.

The term “conjugate” is used herein according to its broadest definition to mean joined or linked together. Molecules are “conjugated” when they act or operate as if joined.

The expression “effective amount” refers to an amount of an agent (e.g. a proanthocyanidin composition etc.) which is effective for preventing, ameliorating or treating the disorder or condition in question. It is contemplated that the proanthocyanidins of the invention will be useful in slowing down, or stopping, progression of tau aggregation and associated degenerative neurological disorders or in enhancing repair of damaged neuronal cells or tissue and assist in restoring proper nerve function.

The terms “treating”, “treatment” and “therapy” as used herein refer to curative therapy, prophylactic therapy, and preventative therapy. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature.

As used herein, the term “disorder” in general refers to any condition that would benefit from treatment with the to cinnamon proanthocyanidin extracts and/or proanthocyanidin compositions described herein. This includes chronic and acute disorders, as well as those pathological conditions which predispose the mammal to the disorder in question. “Neurological disorder” is used herein to refer to conditions that include neurodegenerative conditions, neuronal cell or tissue injuries characterized by dysfunction of the central or peripheral nervous system or by necrosis and/or apoptosis of neuronal cells or tissue, and neuronal cell or tissue damage associated with trophic factor deprivation. Examples of neurodegenerative diseases include familial and sporadic amyotrophic lateral sclerosis (FALS and ALS, respectively), familial and sporadic Parkinson's disease, Huntington's disease (Huntington's chorea), familial and sporadic Alzheimer's disease, Spinal Muscular Atrophy (SMA), optical neuropathies such as glaucoma or associated disease involving retinal degeneration, diabetic neuropathy, or macular degeneration, hearing loss due to degeneration of inner ear sensory cells or neurons, epilepsy, Bell's palsy, frontotemporal dementia with parkinsonism linked to chromosome 17 (FTDP-17), multiple sclerosis, diffuse cerebral corical atrophy, Lewy-body dementia, Pick disease, trinucleotide repeat disease, prion disorders (e.g. Creutzfeldt-Jacob disease), and Shy-Drager syndrome. Injury or damage of neuronal cells or tissue may occur from a variety of different causes that compromise the survival or proper function of neuronal cells or tissue, including but not limited to: acute and non-acute injury from, e.g., ischemic conditions restricting (temporarily or permanently) blood flow as in global and focal cerebral ischemia (stroke); incisions or cuts for instance to cerebral tissue or spinal cord; lesions or placques in neuronal tissues; deprivation of trophic factor(s) needed for growth and survival of cells; exposure to neurotoxins such as chemotherapeutic agents; as well as incidental to other disease states such as chronic metabolic diseases such as diabetes or renal dysfunction.

By “subject” or “patient” is meant any single subject for which therapy is desired, including humans. Also intended to be included as a subject are any subjects involved in clinical research trials not showing any clinical sign of disease, or subjects involved in epidemiological studies, or subjects used as controls.

The term “mammal” as used herein refers to any mammal classified as a mammal, including humans, cows, horses, dogs and cats. In a preferred embodiment of the invention, the mammal is a human.

“Neuronal cells or tissue” refers generally to motor neurons, interneurons including but not limited to commissural neurons, sensory neurons including but not limited to dorsal root ganglion neurons, dopamine (DA) neurons of substantia nigra, striatal DA neurons, cortical neurons, brainstem neurons, spinal cord interneurons and motor neurons, hippocampal neurons including but not limited to CA1 pyramidal neurons of the hippocampus, and forebrain neurons. The term neuronal cells or tissue is intended herein to refer to neuronal cells consisting of a cell body, axon(s) and dendrite(s), as well as to axon(s) or dendrite(s) that may form part of such neuronal cells.

### Description of Certain Methods, Materials and Mechanistic Aspects of the Invention

Self-association of tau including tau aggregation, is believed to play a role the pathological mechanisms involved in Alzheimer's disease. Hence, there is much interest in finding therapies that could block the formation of abnormal protein-associated states of tau in Alzheimer's disease. Recent work provides evidence important roles for insulin in the brain and a relationship of Alzheimer's disease to diabetes. As noted for example in Josh Fischman's article emphasizing the connection between diabetes and Alzheimer's Disease and the possibility of therapies for treating diabetes may extend to Alzheimer's Disease, posted Jul. 24, 2006 on usnews.com, “But bond also brings hope. The same drugs that successfully treat diabetes may actually forestall the brain disease. “It is preliminary, but it is truly exciting, says neurologist Ronald Petersen, director of the Mayo Clinic's Alzheimer's Disease Research Center in Rochester, Minn “We've been kind of stuck developing new Alzheimer's therapies, and this gives us a whole new avenue to try.” It has been suggested that Alzheimer's disease is a neuroendocrine disease, and it has been called diabetes type 3. Neuronal death in humans can be caused by defects in insulin signaling in the brain.

Whole cinnamon, or its water-extract have been shown to possess insulin-like activity and purification of the active material and its use with purified proteins and 3T3-L1 cells provide evidence how this material, now identified as a proanthocyanidin, can affect insulin signaling and potentiate the action of insulin. Recent research studies show that cinnamon or its water-extracted material is safe and effective for treating individuals with type 2 diabetes. Numerous studies have demonstrated how important insulin signaling is in the brain for normal function and how changes in insulin mediated processes could lead to neurodegeneration. Individuals with type 2 diabetes are more prone to get Alzheimer's disease and changes in tau protein are seen in animals defective in insulin signaling.

Aging can lead to susceptibility of certain diseases like type 2 diabetes and Alzheimer's disease. Cinnamon and its bioactive proanthocyanidins in extracts have been found safe and effective for treating diabetes. Because of these studies, cinnamon, cinnamon extract, or proanthocyanidin compositions found therein may have uses not only for long term treatment of type 2 diabetes but also provide extra benefit to type 2 diabetics and others by preventing or slowing down the progression of Alzheimer's disease. Experiments noting that related compounds can get into the brain and inhibit neurodegeneration provides evidence that compounds from cinnamon are useful for the treatment of Alzheimer's disease. Cinnamon proanthocyanidins are known to bind strongly to some disordered proteins rich in proline or proteins like collagen, which have repeat structures involving proline residues. They are also known to bind avidly to unstructured but not to globular proteins. Tau is a disordered protein with a proline rich region and with other regions containing a repeat structure containing proline. As disclosed herein, cinnamon extracts and proanthocyanidins from cinnamon can interact with tau and alter its aggregation, a process associated with formation of tangles described in Alzheimer's disease. For example, water extracts of cinnamon, solids obtained from these extracts containing a mixture of proanthocyanidins, or a proanthocyanidin type A trimer, can inhibit tau aggregation.

As disclosed herein, aggregation of tau, a process associated with Alzheimer's disease, can be inhibited by substances derived from cinnamon as determined by a number of complementary assays including fluorescence, light scattering measurements, gel electrophoresis, and electron microscopy. Because no adverse effects of cinnamon extracts have been observed in human trials to treat diabetes, the disclosure provided herein provides evidence that cinnamon extracts solids containing its bio-active proanthocyanidins may be useful in therapeutic methods to prevent or slow down the progression of tauopathies such as Alzheimer's disease. Embodiments of the bioactive compositions described herein are typically water-soluble extract derived from a natural source—cinnamon. The relevant phytochemicals contained in the extract are a class of flavanol compounds known as proanthocyanidins, which are polymers of epicatechin—the basic building block of EGCG, the purported health-beneficial compound present in green tea. While the general health-promoting effects of flavanoids are widely attributed to their associated antioxidant activities, the tau aggregation inhibitory activity of proanthocyanidins appears to be distinct from their antioxidant properties. Cinnamon from Ceylon contains several proanthocyanidin species, the most prevalent being a specific trimer molecule, which in purified form displays inhibitory activity on its own. The extract, however, is significantly more potent than the purified trimer, implicating the presence of other molecules in the extract that are active. Cinnamon is easily extracted in large quantities as a liquid, and then freeze-dried to a powder which can be stored or encapsulated for oral administration. A similar extract has been tested for treatment of type II diabetes, and appears to be safe for human consumption.

While studies have suggested that epigallocatechin gallate of green tea or a synthetic proanthocyanidin dimer B1 (not found in cinnamon) could effectively inhibit tau aggregation, no published reports were found describing the use of cinnamon extracts containing a mixture of proanthocyanidin compounds (e.g. proanthocyanidin A trimer) showing that these materials interact with tau directly and influence its biological properties. Interestingly, there may be a connection between impaired insulin action and phosphorylation of tau, a process associated with tau aggregation in Alzheimer's disease, as well as a connection between insulin action and neuronal death. A report has been published showing that those with type 2 diabetes were more prone to get Alzheimer's disease. Over the last 10 years, considerable evidence points to a common pathology between Alzheimer's disease and type II diabetes. For example, abundant literature describes insulin resistance and diabetes as significant risk factors for AD. In particular, diabetics incur an approximate 65-70% increased risk of developing AD compared to non-diabetic controls, and such individuals display increased amyloid plaques and neurofibrillary tangle load in the hippocampus at autopsy. However, no work has been done that shows that the proanthocyanidins with insulin-like activity from cinnamon can affect a key process, the formation of tangles of tau, associated with Alzheimer's disease. The disclosure provided herein provides evidence that cinnamon and its proanthocyanidins, compounds shown to be helpful to type 2 diabetics may slow the progression of Alzheimer's disease.

In this context, embodiments of the present invention relates to the disclosure provided herein that proanthocyanidins (e.g. those derived from cinnamon extracts): (1) can bind to tau as seen by the observation that spectral characteristics of proanthocyanidins are perturbed by tau, (2) can inhibit the aggregation of tau as determined by light scattering studies, (3) can inhibit tau aggregation detected by fluorescence measurements, (4) preserve soluble tau from aggregation as determined by gel electrophoresis and (5) affect the formation of filaments as assessed by electron microscopy. Aggregation of tau also is potently inhibited by a water extract of cinnamon containing proanthocyanidins showing that an extract alone can be very effective of this process. A procedure for extraction of cinnamon powder and a method for obtaining 100% water-soluble solids is also described herein. In addition, it has been found that proanthocyanidin A does not inhibit the normal function of tau, microtubule assembly. Because of this result and the effect of proanthocyanidin on a process associated with pathology of the disease, cinnamon proanthocyanidin or an extract of cinnamon containing these bio-active compounds may be useful in the treatment of Alzheimer's disease.

In addition to tangle formation, abnormal hyperphosphorylation of PHF-tau at multiple phosphorylation sites is classically correlated with AD and the phosphorylation by CdK5 is considered an important event associated with pathology of this syndrome. Among several protein kinases that can phosphorylate tau in vitro, cdk5/p25 is believed to be crucial. While the precise role of tau phosphorylation in neurodegeneration remains unclear, the overexpression of cdk5/p25 has been shown to cause neurofibrillary pathology and neurodegeneration. Thus in addition to tau aggregation, the inhibition of cdk5/p25 by small molecule inhibitors may represent an important strategy for possible therapy. As described herein, cdk5/p25 kinase activity is potently inhibited by cinnamon extract in vitro. Given the role of cdk5 kinase activity in tau phosphorylation and in pancreatic insulin secretion, inhibition of this enzyme may have complementary beneficial effects for diabetes, first in restoring insulin secretion from the pancreas, and secondly in slowing the neurodegeneration and cognitive decline to which diabetic patients are predisposed.

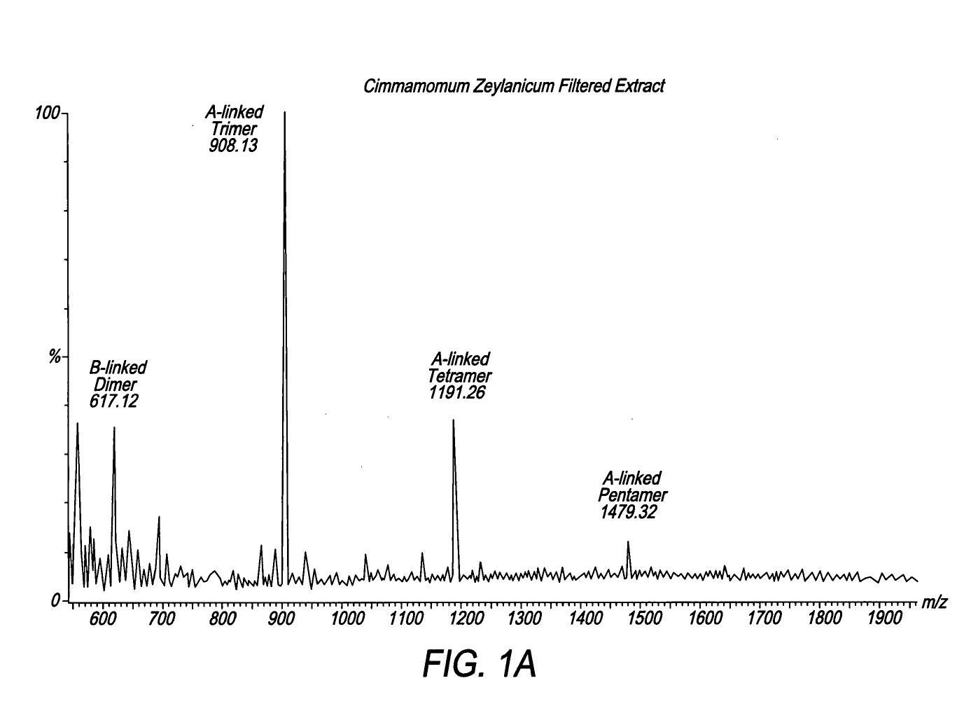

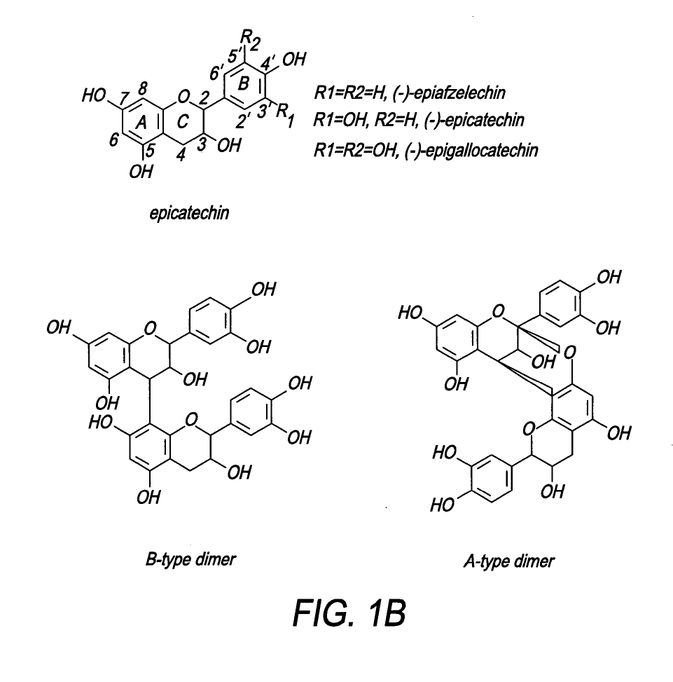

The disclosure provided herein demonstrates the inhibitory effects of cinnamon extracts and the proanthocyanidins contained therein on tau aggregation. As is known in the art, proanthocyanidins are polyphenols derived from epicatechin or related flavonoids by covalent linkage forming oligomers of 2, 3, 4, 5, 6, 7, 8, 9 or 10 monomeric units or more. Cinnamon extract (CE) contains multiple proanthocyanidin species (FIG. 1a) whose monomeric units are singly- or doubly-linked through C—C or C—O bonds, giving rise to the quinol (B-type) or quinone (A-type) redox forms, respectively (FIG. 1b). Among the various cinnamon species that are commercially available, Cinnamomum zeylanicum from Ceylon contains mainly A-type trimer along with some B-type dimer, while Cinnamomum cassia from China contains mainly B-type dimer with some B-type trimer. Experiments disclosed herein use for example aqueous extracts from C. zeylanicum or, alternatively, the A-type trimer purified from this extract.

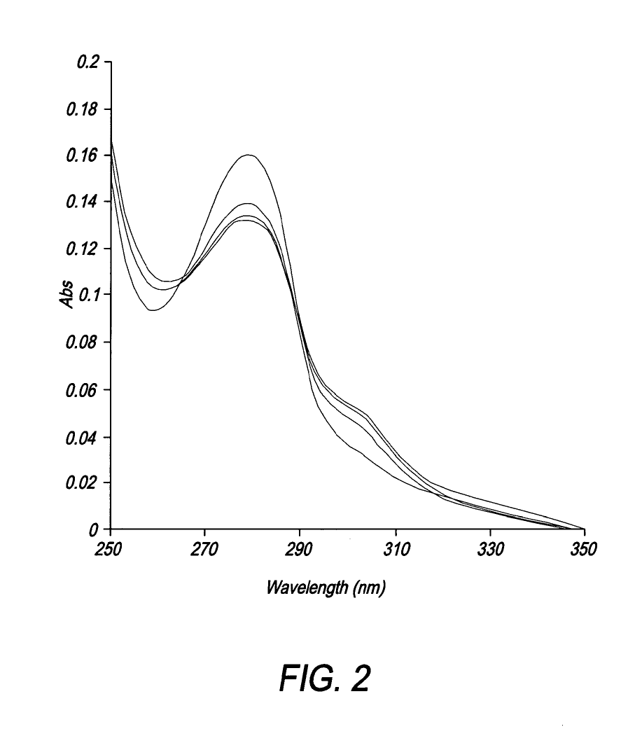

As shown by the data disclosed in the various figures, proanthocyanidins bind to tau and inhibit tau aggregation. In illustrative experiments, full-length tau and an engineered truncation fragment of tau (tau187) were generated by over-expression of the recombinant human protein in bacteria followed by purification to homogeneity by conventional chromatography. Purified A-type trimer displays affinity for monomeric tau based on the change in its UV absorbance spectrum seen with increasing concentrations of tau187 protein (see, e.g. the data shown in FIG. 2). The UV spectra display an isosbestic point consistent with a specific binding mechanism. As further described herein, CE and purified proanthocyanidins can inhibit tau fiber formation in an in vitro aggregation assay. In these experiments, heparin-induced aggregation of tau into PHF-like fibers is routinely monitored by four methods: 1) enhanced Thioflavin S fluorescence emission; 2) increased turbidity measured by OD350; 3) ultracentrifugation of sarkosyl-treated samples, and 4) electron microscopy. The disclosure provided herein shows that tau aggregated into fibers over a time course typically on the order of minutes to hours (FIG. 3a), and that these fibers were typical of PHFs isolated from AD brain (FIG. 3c). However, in the presence of C. zeylanicum extract or purified proanthocyanidin A trimer, aggregation was dramatically inhibited (FIG. 3a) resulting in short fibers that were very much less abundant (FIG. 3d). To ensure that proanthocyanidin molecules were not simply binding to and precipitating tau in a non-specific manner, the amount of tau that fractionated with the pellet vs. supernatant after high-speed ultracentrifugation was analyzed. In the absence of proanthocyanidin A trimer, tau aggregation resulted in a significant amount of tau in the pellet fraction. But, in the presence of 200 uM trimer, aggregation was inhibited and tau was found to remain in the soluble fraction (FIG. 3b). This was also true in response to whole CE (C. zeylanicum), which displayed inhibitory activity in a dose-dependent manner (FIG. 4).

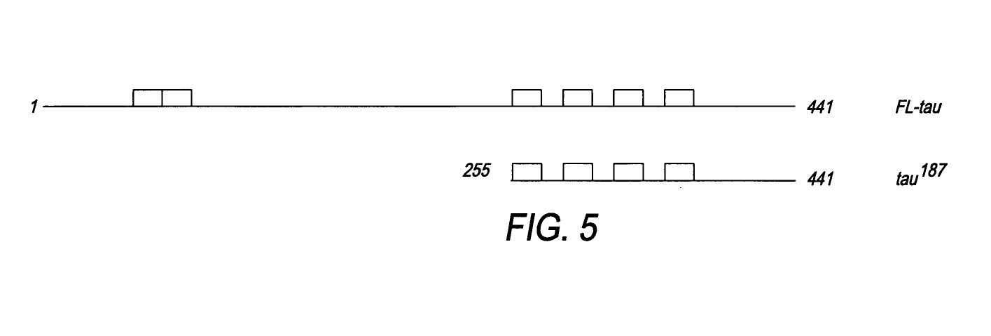

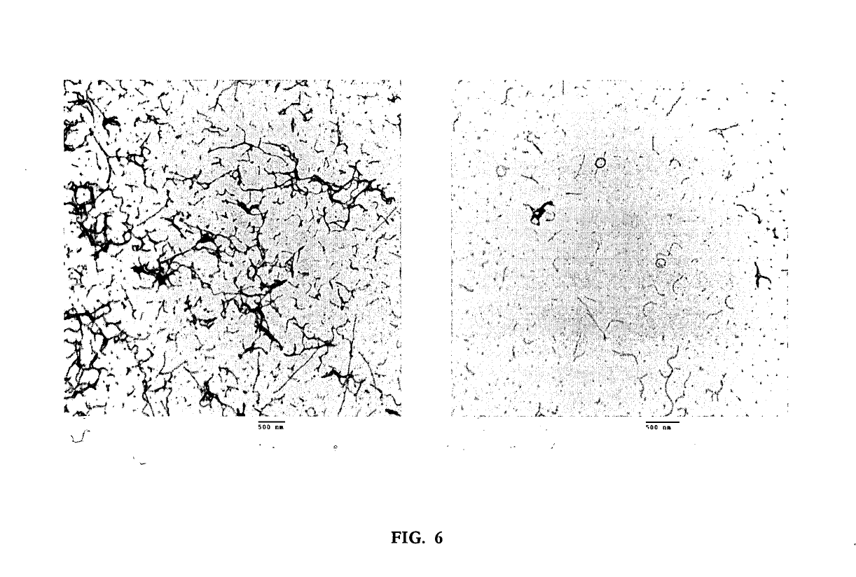

Two hexapeptide motifs in tau are critical for tau aggregation and fiber formation (see, e.g. von Bergen et al., Proc Nad Acad Sci USA 97, 5129-34 (2000)). These motifs lie in the first and second inter-repeat regions of the MT binding domain (FIG. 5), and it is known that engineered truncation fragments of tau that contain these motifs often undergo aggregation into fibers even more efficiently than the full-length molecule (see, e.g. Barghorn et al., Biochemistry 41, 14885-96 (2002)). In this context, tau187 is a truncation fragment of full-length tau that was constructed to include the essential motifs known to be involved in tau aggregation and in this way facilitate the characterization of data resulting from certain studies disclosed herein. Tau187 comprises four 31 amino acid repeat sequences responsible for binding microtubules (MT) in vivo, and the C-terminal tail (FIG. 5). Like full-length tau, tau187 aggregates into long fibers as examined by EM (FIG. 6a). In the presence of CE or purified trimer, aggregation was effectively inhibited, as seen by fibers that were short and few in number (FIG. 6b). Tau187 in our hands has proven to aggregate more consistently and reproducibly than FL tau, and consequently has served as the most reliable system of fiber formation.

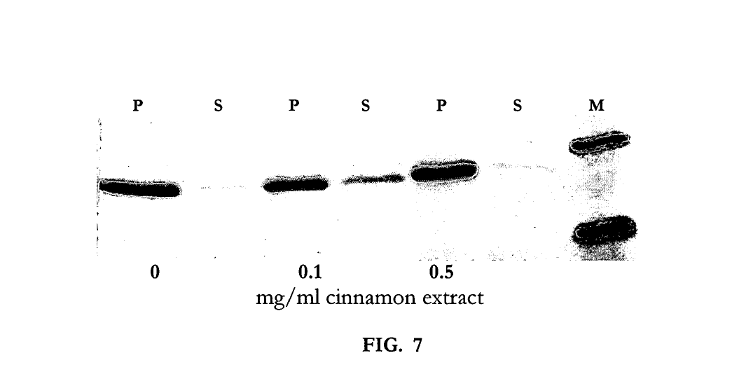

Since tau aggregation is routinely initiated by heparin, the possibility of the inhibitory effect of the CE on aggregation being due to interference of the heparin-tau interaction was tested. FIG. 7 shows that tau187 stably interacts with heparin-Sepharose and this interaction is not disrupted by CE at high concentrations (five-fold higher than that routinely employed). Thus, this disclosure provides evidence that the inhibition of tau aggregation by CE or proanthocyanidins is through a specific effect on tau, and occurs via a specific mechanism as opposed to a non-specific manner.

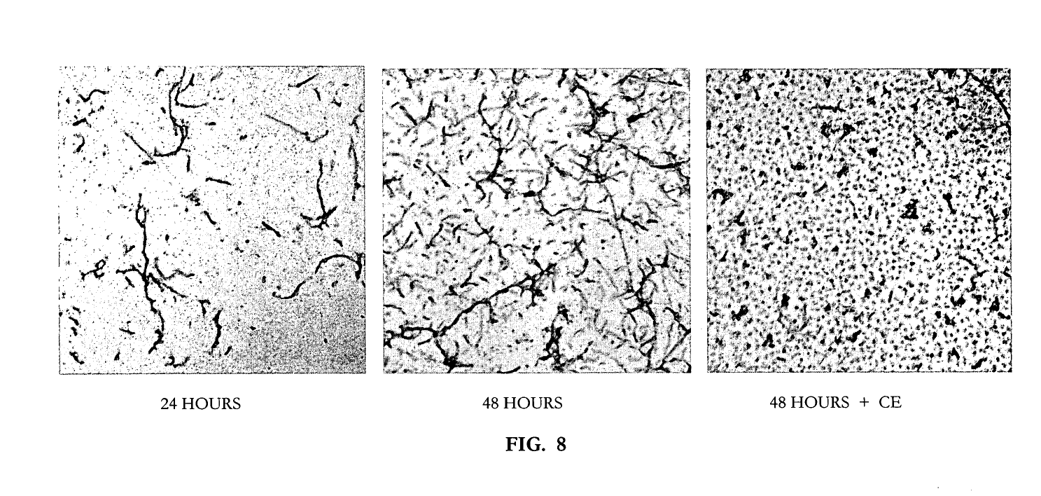

As described herein CE can disassemble pre-existing fibers. Tau187 was induced to undergo aggregation for 24 hrs at which time fibers were treated with either extract or water, followed by incubation for another 24 hrs. In these studies, pre-existing fibers were significantly dissolved in the presence of CE, whereas fibers continued to accumulate in the control sample (FIG. 8). This provides evidence that not only might CE prevent de novo fiber growth, but further reverses neurofibrillary tangles observed with various neurological pathologies in vivo.

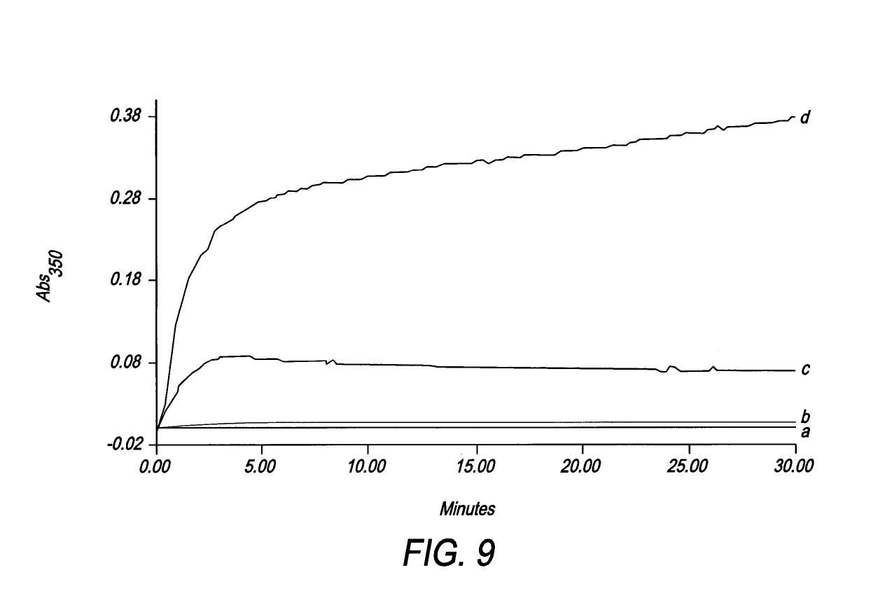

The normal function of tau in neurons is to regulate MT assembly and dynamics. Thus it is desirable for a potential AD therapeutic to inhibit tau aggregation but not impede normal tau function. To test this, purified tubulin was incubated with either CE, tau187 or both, and MT assembly was monitored by solution turbidity. FIG. 9 shows that, like FL-tau, tau187 promotes MT assembly from free tubulin, and CE does not impede this function. In fact, tau-dependent MT assembly was actually enhanced by CE. The reason for this is unclear, but since tau is normally an unstructured molecule, it is possible that CE promotes a conformer in tau that favors MT binding. Thus CE and proanthocyanidins appear to act as specific inhibitors of tau aggregation that are free of side effects on normal neuron function.

The disclosure provided herein also shows that the disclosed cinnamon extracts inhibit the phosphorylation of tau by cdk5/p25 kinase. Specifically, in addition to tangle formation, abnormal hyperphosphorylation of PHF-tau at multiple phosphorylation sites is classically correlated with AD (see, e.g. Buee et al., Brain Res Brain Res Rev 33, 95-130 (2000); and Lovestone et al., Neuroscience 78, 309-24 (1997)). The relevant phosphorylation sites in PHF-tau isolated from AD brain have been characterized by mass spectrometry analysis which reveals as many as 19-25 sites of phosphorylation (see, e.g. Hanger et al., J Neurochem 71, 2465-76 (1998); Hasegawa et al., J Biol Chem 267, 17047-54 (1992); and Morishima-Kawashima et al., J Biol Chem 270, 823-9 (1995)). Among several protein kinases that can phosphorylate tau in vitro, cdk5/p25 is believed to be crucial (see, e.g. Imahori et al., Neurobiol Aging 19, S93-8 (1998)) and overexpression of cdk5/p25 has been shown to cause neurofibrillary pathology and neurodegeneration (see, e.g. Nishimura et al., Cell 116, 671-82 (2004); Noble et al., Neuron 38, 555-65 (2003); Jackson et al., Neuron 34, 509-19 (2002); and Cruz et al., Neuron 40, 471-83 (2003)). Thus the methods and materials disclosed herein for the inhibition of cdk5/p25 represents another important strategy for tauopathy disease therapy.

Cdk5/p25 is a neural-specific cyclin-dependent kinase originally discovered by our laboratory (see, e.g. Lew et al., J Biol Chem 267, 13383-90 (1992); Lew et al., J Biol Chem 267, 25922-6 (1992); and Lew et al., Nature 371, 423-6 (1994)), and independently as tau protein kinase 2 by Imahori et al (see, e.g. Uchida et al., FEBS Lett 355, 35-40 (1994); and . Ishiguro et al., FEBS Lett 342, 203-8 (1994)). The p25 subunit is essential for cdk5 kinase activity (see, e.g. Qi et al., J Biol Chem 270, 10847-54 (1995)) and is generated by N-terminal cleavage of the full-length version of this molecule, p35 (see, e.g. Lew et al., Nature 371, 423-6 (1994); and Tsai et al., Neuron 18, 29-42 (1997)). While p35 is essential for normal cortical development (see, e.g. Chae, T. et al., Neuron 18, 29-42 (1997)), p25 is associated with tau hyperphosphorylation, neurofibrillary pathology, and neuron death (see, e.g. Noble et al., Neuron 38, 555-65 (2003); and Cruz et al., Neuron 40, 471-83 (2003)). Conversion of p35 to p25 has been linked to AD (see, e.g. Patrick et al., Nature 402, 615-22 (1999); and Patrick et al., Nature 411, 764-765 (2001)) and can occur in response to a variety of stresses including exposure of cells to Aβ peptide (see, e.g. Lee et al., Nature 405, 360-4 (2000)), the protein involved in formation of the senile plaques of AD.

P35 is classically a neural-specific protein (see, e.g. Lew et al., Nature 371, 423-6 (1994); and Tsai et al., Neuron 18, 29-42 (1997)). However, its expression has recently been shown to be upregulated in β-cells of the pancreas in response to chronic high glucose exposure (see, e.g. Ubeda et al., J Biol Chem 281, 28858-64 (2006)). The resulting increase in cdk5/p35 activity is essential in mediating the down-regulation of the insulin gene, a classical symptom of glucose toxicity and a critical component of the pathophysiology of type II diabetes. In this system, inhibitors of cdk5/p35 restored insulin secretion. The novel role of cdk5 in insulin expression provides a further link between AD and diabetes. Given the role of cdk5 kinase activity in tau phosphorylation and now in pancreatic insulin secretion, inhibition of this enzyme may have complementary beneficial effects for diabetes, first in restoring insulin secretion from the pancreas, and secondly in slowing the neurodegeneration and cognitive decline to which diabetic patients are predisposed.

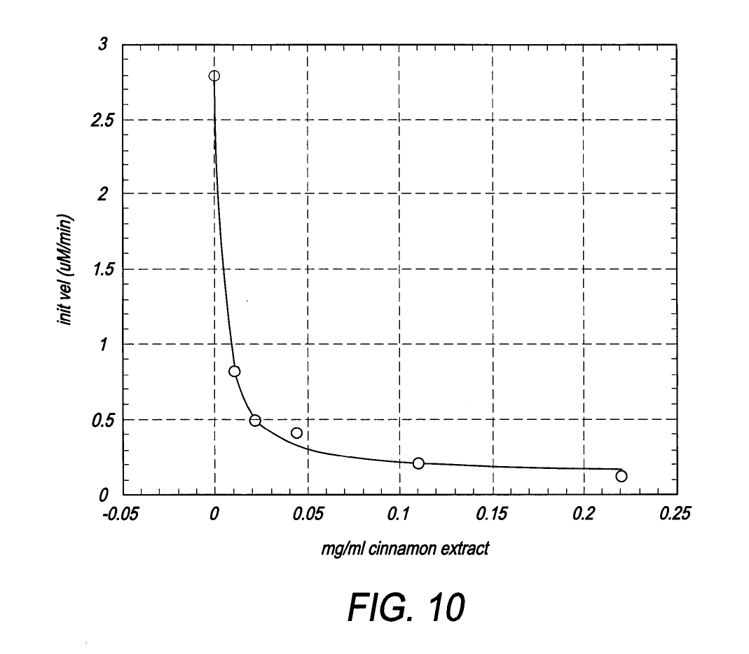

As noted above, cdk5/p25 kinase activity is potently inhibited by cinnamon extract in vitro (FIG. 10). The inhibition follows Michaelis-Menten dose-dependency when both tau and histone H1 are employed as substrates. Preliminary experiments reveal IC50 values of approximately 3.6 and 13 ug/ml of extract for the phosphorylation of histone H1 (FIG. 10) and tau, respectively. The fact that inhibition is seen with different substrates provides evidence that the inhibitor acts by targeting the cdk5/p25 complex itself, as opposed to the protein substrate.

As discussed below, embodiments of the invention include novel compositions for binding tau and/or reducing and/or inhibiting tau aggregation. Typically these compositions comprise water extracts of Cinnamomum zeylanicum, Cinnamomum cassia, Cinnamomum loureirii as well as proanthocyanidins from cinnamon or can be derived from other plant species that are obtained using other methods known in the art. One embodiment of the invention disclosed herein comprises a method of inhibiting and/or reducing the ability of tau to aggregate comprising exposing tau to a proanthocyanidin, wherein the proanthocyanidin binds to tau thereby inhibiting aggregation. In this context, the disclosure provided herein teaches that cinnamon extracts can cause the depolymerization of tau aggregates formed in the presence of heparin alone and further that cinnamon extract or proanthocyanidin A does not interfere with a normal function of tau, stimulation of the formation of microtubules. Moreover, the disclosure provided herein shows that cinnamon extracts inhibits Cdk5, a protein kinase, that phosphorylates tau and which is suggested to be involved in the pathologies associated with Alzheimer's disease.

### Illustrative Embodiments of the Invention

Embodiments of the present invention relates to the disclosure provided herein that proanthocyanidins (e.g. those derived from cinnamon extracts) can bind to tau, can inhibit tau aggregation and can protect soluble tau from aggregation. In this context, embodiments of the invention include proanthocyanidin compositions as well as methods for making and using such compositions. In an illustrative embodiment of the invention, the disclosure provides methods for using isolated proanthocyanidins to bind tau isoforms (e.g. to identify the presence of and/or characterize the aggregation status of and/or chromatographically separate tau polypeptides). Optionally these methods further inhibit or reduce the formation of tau aggregates, a phenomenon observed in neurodegenerative diseases such as Alzheimer's disease. Typically in these methods, the tau polypeptide comprises tau-A (SEQ ID NO: 1), tau-B (SEQ ID NO: 2), tau-C (SEQ ID NO: 3), tau-D (SEQ ID NO: 4), tau-E (SEQ ID NO: 5), tau-F (SEQ ID NO: 6) or tau-G (SEQ ID NO: 7) or a proteolytically processed fragment thereof.

The invention disclosed herein has a number of embodiments. One embodiment of the invention is a method of binding a mammalian tau polypeptide with an isolated proanthocyanidin by combining the tau polypeptide with an isolated proanthocyanidin composition and then allowing an isolated proanthocyanidin in the composition to bind the tau polypeptide so that the tau polypeptide is bound by the isolated proanthocyanidin. Of course those of skill win the art will understand that this binding is carried out under reaction conditions (e.g. physiological conditions or those that approximate physiological conditions) and for a period of time sufficient for the binding to occur (as can readily be determined using the methods and materials disclosed herein. In typical embodiments of this method, proanthocyanidin(s) bound to the tau polypeptide can be used to observe the presence of tau polypeptides in a biological sample (e.g. an aggregation of tau polypeptides in a biological sample). As will be understood by those of skill in this art, in such methods, observation of an absence of a proanthocyanidin/tau binding complex correspondingly provides evidence that tau polypeptides are not present in this biological sample. These binding methods can be used in a variety of contexts. For example, embodiments of these methods can include using observations of the presence of an aggregation of tau polypeptides to diagnose a tauopathy (e.g. Alzheimer's disease).

In certain embodiments of the invention, binding of tau polypeptide by an isolated proanthocyanidin can be used to perturb (e.g. inhibit) the ability of the tau polypeptide to form an aggregation of tau polypeptides. Optionally, such methods can be performed in vivo in a mammal having a neurological condition or disorder. In an illustrative example of this, the binding of the tau polypeptide by the isolated proanthocyanidin can be used to perturb the ability of tau polypeptides to form an aggregation in an individual suffering from a tauopathy. Certain embodiments of the methods designed to inhibit tau polypeptide aggregation include the step of observing a perturbation in tau polypeptide aggregation after the tau polypeptide is combined with the proanthocyanidin composition. For example, one can directly observe a perturbation in tau polypeptide aggregation after the tau polypeptide is combined with the proanthocyanidin composition by using for example a thioflavin T assay and/or via electron microscopy. Alternatively, one can indirectly observe a perturbation in tau polypeptide aggregation after the tau polypeptide is combined with the proanthocyanidin composition by comparing the behavior on a mammal exhibiting a tauopathy treated with the proanthocyanidin composition with that of an untreated control. In this context, a variety of behavior tests designed for such comparative purposes, for example the Morris water maze or object recognition tests discussed below.

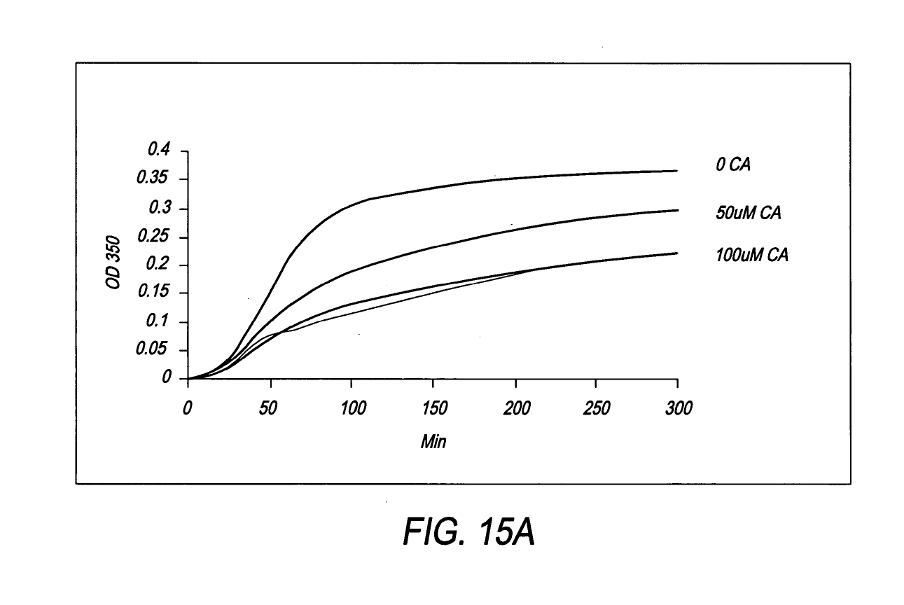

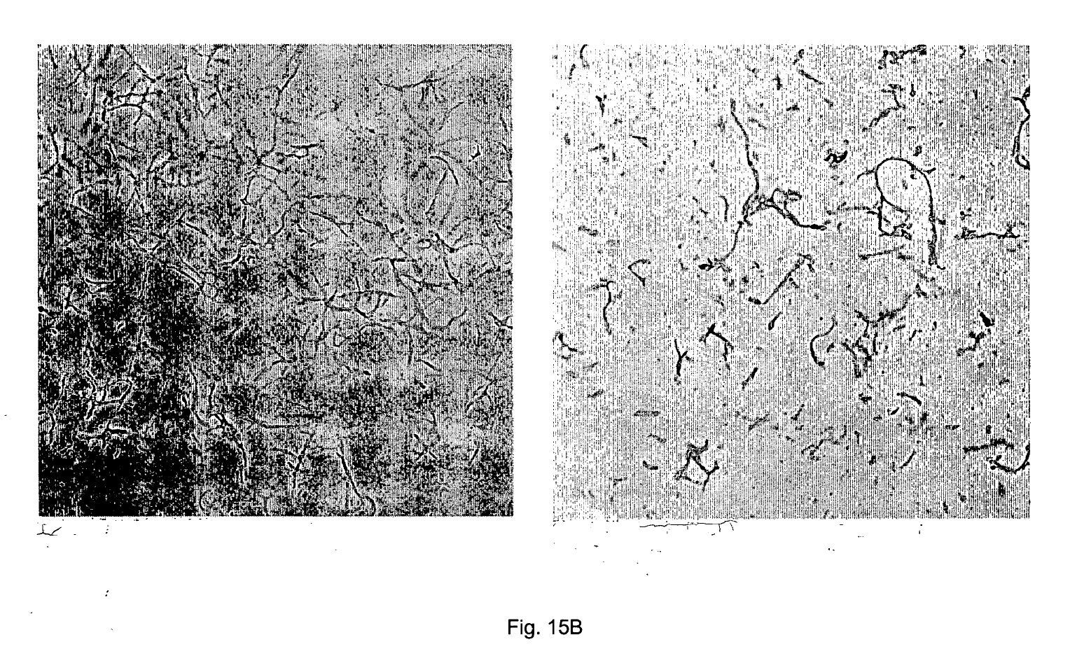

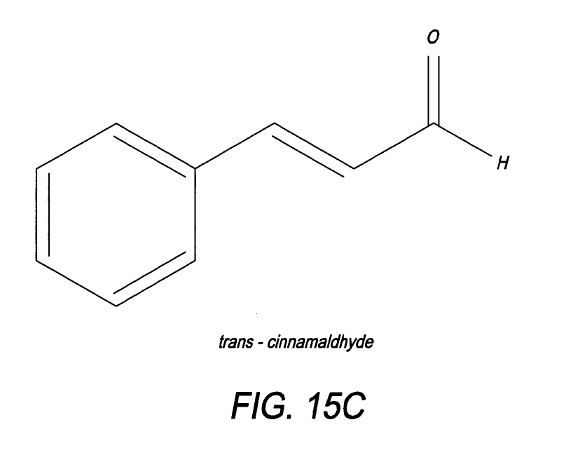

Another illustrative embodiment of the invention is a method of treating a mammal having a neurological condition or disorder characterized by an aggregation of tau polypeptides (e.g. Alzheimer's disease), comprising administering to said mammal an effective amount of an isolated proanthocyanidin composition selected for its ability to perturb aggregation (e.g. inhibit the formation of new aggregates or dissociate existing aggregates) of tau polypeptides. Optionally in these methods, the isolated proanthocyanidin composition comprises at least one of an A-linked proanthocyanidin dimer, trimer, tetramer, or pentamer; a proanthocyanidin B2; a cinnamaldehyde (see, e.g. FIG. 15); an oxidized catechin; or an oxidized epicatechin.

Yet another embodiment of the invention is a method determining if a proanthocyanidin compound binds to a tau polypeptide comprising combining the tau polypeptide with a proanthocyanidin compound and then testing the combination of the tau polypeptide and the proanthocyanidin compound to determine if the proanthocyanidin compound binds the tau polypeptide. Optionally in such methods, the proanthocyanidin compound is coupled to a detectable marker. Proanthocyanidin compounds coupled a detectable marker such as a chromogenic marker, a fluorescent tag, a radiolabel, a magnetic tag, or an enzymatic reaction product etc. may for example allow the proanthocyanidin's binding to tau polypeptides to be more readily observed.

A related embodiment of the invention is a method of determining if an isolated proanthocyanidin compound perturbs aggregation of tau polypeptides comprising observing the ability of tau polypeptides to aggregate in the absence of the isolated proanthocyanidin compound; combining tau polypeptides with the isolated proanthocyanidin compound and observing the ability of tau polypeptides to aggregate in the presence of the isolated proanthocyanidin compound, wherein a decrease in tau aggregation observed in the presence of the isolated proanthocyanidin compound as compared to the amount of tau aggregation observed in the absence of the isolated proanthocyanidin compound identifies the isolated proanthocyanidin compound as perturbing the ability of tau polypeptides to aggregate.

As discussed in detail below, the proanthocyanidin compositions used in embodiments can be obtained from a variety of sources. Typically, however, the isolated proanthocyanidin composition is derived from Cinnamomum loureirii, Cinnamomum cassia or Cinnamomum zyelanicum. Optionally, the isolated proanthocyanidin composition is derived from an aqueous extract of these Cinnamomum species and comprises at least one of the following compounds: an A-linked proanthocyanidin dimer, trimer, tetramer, or pentamer; a proanthocyanidin B2; a cinnamaldehyde; an oxidized catechin; or an oxidized epicatechin. In certain embodiments of the invention, the proanthocyanidin composition comprises at least the A-linked proanthocyanidin trimer.

Another embodiment of a proanthocyanidin composition of the invention comprises isolated proanthocyanidin compound derived from Cinnamomum loureirii, Cinnamomum cassia or Cinnamomum zyelanicum and characterized by at least one of the following properties: an ability to bind tau polypeptides; an ability to reduce the ability of tau polypeptides to form an aggregation as observed by a thioflavin T assays; an ability to reduce the number and length of tau filament formation as measured by electron microscopy studies, exhibits a decrease in the absorbance spectra upon combination with tau polypeptides; or comprises: an B-linked proanthocyanidin dimer having a mass spectroscopy peak of 617 m/z; an A-linked proanthocyanidin trimer having a mass spectroscopy peak of 905 m/z; an A-linked proanthocyanidin tetramer a mass spectroscopy peak of 1193 m/z; or an A-linked proanthocyanidin pentamer having a mass spectroscopy peak of 1481 m/z; a proanthocyanidin B2; a cinnamaldehyde; an oxidized catechin; or an oxidized epicatechin. In describing compounds such as linked proanthocyanidin trimer etc., those of skill in the art understand that this language is intended to encompass these compounds as well as the salts of these compounds (e.g. pharmaceutically acceptable salts known in the art). For example, as is known in the art, many compounds can occur both a free acid form as well as sodium, potassium or ammonium salts, and other salts derived from alkaline earth elements or other metallic salts.

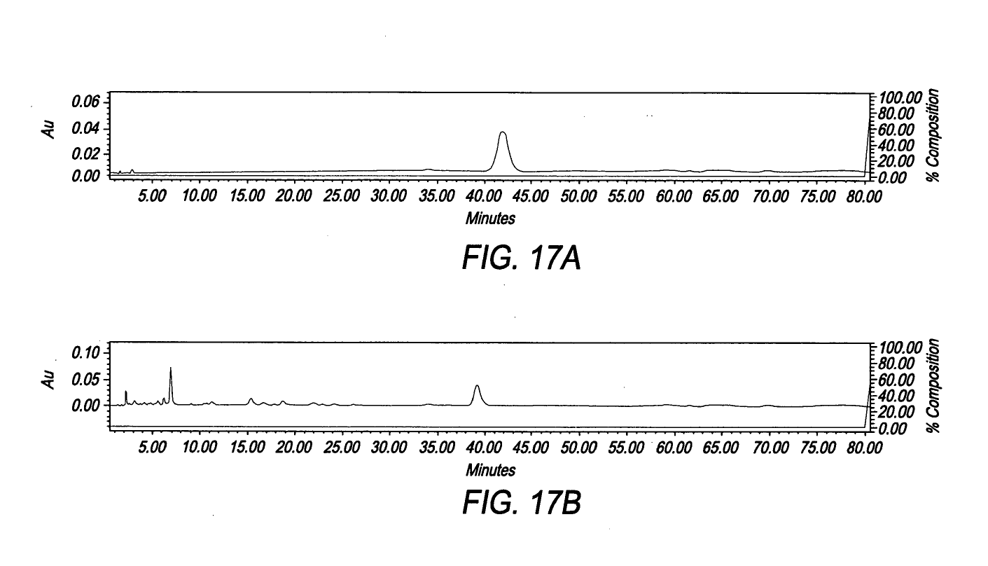

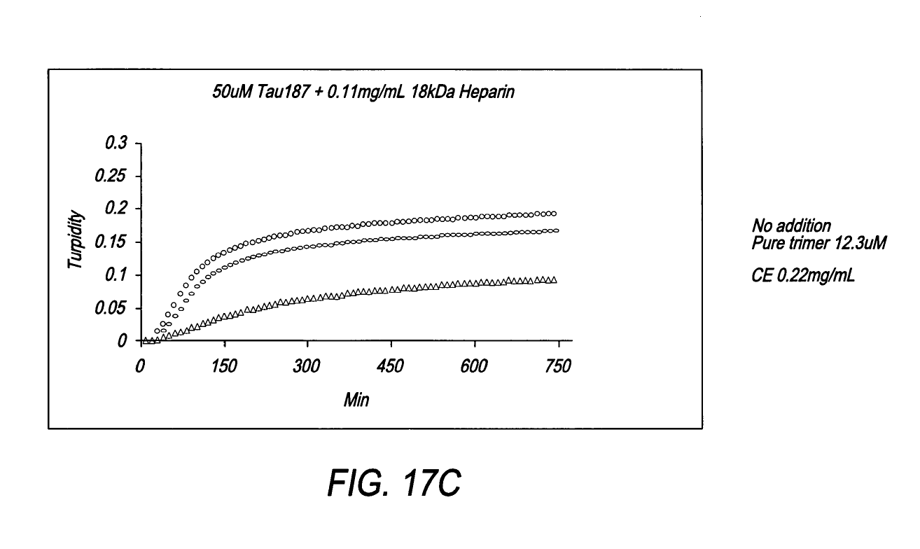

The data shown in FIG. 17 provides evidence both that a purified single species of proanthocyanidin (proanthocyanidin A-type trimer) inhibits tau polypeptide aggregation and further that a whole cinnamon extract proanthocyanidin composition inhibits tau polypeptide aggregation more potently than does this purified proanthocyanidin A-type trimer alone. This data provides evidence that a plurality of compounds within the whole cinnamon extract proanthocyanidin composition are working in an additive (and perhaps synergistic) manner to inhibit tau polypeptide aggregation. Consequently, embodiments of the invention include proanthocyanidin composition having a plurality of compounds present in the cinnamon extracts disclosed herein, for example both proanthocyanidin A-type trimer as well as one or more additional compounds such as other proanthocyanidin A-type multimers as well as other compounds such as proanthocyanidin B2; a cinnamaldehyde; an oxidized catechin; an oxidized epicatechin or the like. In this context, those of skill in the art will readily understand that the methods disclosed herein allow one to identify the various compounds that contribute to the additive or synergistic effect observed in FIG. 17 without undue experimentation. For example, one of skill in the art can simply subject the whole cinnamon extract proanthocyanidin composition to a series of further purification steps while observing the aggregation inhibiting activity of each purified faction that results from such steps. In this context, when the additive or synergistic effect observed in FIG. 17 is lost, the compound(s) of interest will then be known to localized to a specific fraction. Such steps can be performed repeatedly until the exact compound is identified. Alternatively, one can make combinations of various purified compounds (e.g. proanthocyanidin A-type trimer as well as one or more additional compounds such as other proanthocyanidin A-type multimers as well as other compounds such as proanthocyanidin B2; a cinnamaldehyde) and test them an assay such as that shown in FIG. 17 to identify those compounds that contribute to the additive or synergistic effect observed in FIG. 17

A discussed in detail below, embodiments of the invention include methods for making the proanthocyanidin compositions disclosed herein. One illustrative embodiment of this is a process for preparing an isolated proanthocyanidin composition comprising: extracting a sample of Cinnamomum loureirii, Cinnamomum cassia or Cinnamomum zyelanicum with an aqueous solution; agitating this extract for at least one minute at a temperature between 40-90° C.; centrifuging the agitated extract so as to produce a first pellet and a first supernatant; incubating the first supernatant of at 0-4° C. for at least one minute; centrifuging the incubated supernatant so as to produce a second pellet and a second supernatant; and then filtering the second supernatant, wherein the resultant filtered second supernatant comprises an aqueous solution of the isolated proanthocyanidin composition. Optionally the method further comprises lyophilizing the aqueous solution of the isolated proanthocyanidin composition so as to form a solid isolated proanthocyanidin composition. Further process steps are considered herein. For example, one can further add an aqueous solution to the solid isolated proanthocyanidin composition noted above so as to form an aqueous solution of the isolated proanthocyanidin composition. Embodiments of the invention further include an isolated proanthocyanidin composition product produced by such processes. A related embodiment of this invention is an isolated proanthocyanidin composition product having the constellation of active proanthocyanidin compounds that are present in the isolated proanthocyanidin composition produced by the processes disclosed herein.

Other related embodiments of the invention include methods for the preparation of a medication for the treatment of tauopathy (e.g. Alzheimer's disease) by preparing a proanthocyanidin composition for administration (typically by combining the composition with a pharmaceutical carrier) to a mammal having the pathological condition. A related method is the use of an effective amount of a proanthocyanidin composition in the preparation of a medicament for the treatment of a tauopathy. Another related method is the use of an effective amount of a cinnamon proanthocyanidin extract in the preparation of a medicament for the treatment of a tauopathy. Yet another related embodiment is a use of a proanthocyanidin composition manufacture of a medicament for perturbing tau aggregation in a patient. Such methods typically involve the steps of including an amount of a proanthocyanidin composition or cinnamon proanthocyanidin extract sufficient to inhibit tau aggregation in vivo and an appropriate amount of a physiologically acceptable carrier. As is known in the art, optionally other agents can be included in these preparations.

Another method of the invention is a method of identifying a proanthocyanidin compound that binds tau and/or reduces the ability of tau to aggregate comprising observing the ability of tau to aggregate in the absence of the proanthocyanidin compound, observing the ability of tau to aggregate in the presence of the proanthocyanidin compound, wherein a decrease in tau aggregate observed in the presence of the proanthocyanidin compound as compared to the amount of tau aggregate observed in the absence of the proanthocyanidin compound identifies the proanthocyanidin compound as binding tau and reducing the ability of tau to aggregate. A related method of the invention is a method of identifying a compound within a cinnamon extract that that binds tau and/or reduces the ability of tau to aggregate comprising observing the ability of tau to aggregate in the abience of the cinnamon extract, observing the ability of tau to aggregate in the presence of the cinnamon extract, wherein a decrease in tau aggregate observed in the presence of the cinnamon extract as compared to the amount of tau aggregate observed in the absence of the cinnamon extract identifies a compound within the cinnamon extract as binding tau and reducing the ability of tau to aggregate. In some embodiments, the method comprises inducing the formation of tau aggregate by the addition of heparin.

Yet another embodiment of the invention is a method of inhibiting the ability of tau to aggregate comprising exposing tau to a proanthocyanidin composition, wherein said exposure inhibits the ability of tau to aggregate. In preferred embodiments of the method said exposure inhibits the ability of tau to form filaments. In highly preferred embodiments, said inhibition of the ability of tau to form filaments is observed using electron microscopy. The methods can further comprise using electron microscopy to observe said binding inhibition.

Another embodiment of the invention is a method of inhibiting the ability of tau to aggregate comprising exposing tau to a proanthocyanidin, wherein the proanthocyanidin binds to tau upon said exposure, thereby inhibiting the ability of tau to aggregate. Typically, said binding perturbs spectral characteristics of the proanthocyanidin. In preferred embodiments, the exposure of tau to the proanthocyanidin causes a decrease in the absorbance of the proanthocyanidin at a wavelength of about 279 nm. Preferably, the proanthocyanidin is a trimer. In highly preferred embodiments, the exposure of tau to the proanthocyanidin causes an isobestic point to appear, wherein the appearance of the isobestic point shows that the proanthocyanidin binds to tau. In certain embodiments, said exposure causes an increase in the absorbance of the proanthocyanidin at 300 nm Typically, said increase represents the ionization of a phenolic group.

Yet another embodiment of the invention is a method of inhibiting the ability of tau to aggregate comprising exposing tau to a cinnamon extract, wherein the cinnamon extract binds to tau upon said exposure, thereby inhibiting the ability of tau to aggregate. In a preferred embodiment, the cinnamon extract comprises a cinnamon A trimer. In a highly preferred embodiment, the cinnamon A trimer is oxidized. In alternate embodiments, the cinnamon A timer is not oxidized. Typically, the method further comprises reducing the light scattering of tau upon said exposure. In these embodiments, the cinnamon A trimer decreases the light scattering and absorbance of tau at a wavelength of 350 nm. Optionally, the greater the concentration of the cinnamon extract, the greater the decrease in light scattering. The decrease in light scattering upon said exposure shows that the cinnamon extract promotes the disaggregated state of tau.

Another embodiment of the invention is a method of reducing the formation of tau fibers comprising exposing tau to a cinnamon extract, wherein the cinnamon extract reduces the ability of tau to form fibers. Preferably, the cinnamon extract comprises a cinnamon A trimer. Typically, the fibers that are formed upon said exposure are of a shorter length than a control sample, wherein the control sample is not exposed to the cinnamon extract. In certain embodiments, the exposure causes an inhibition of tau aggregation as probed by electron microscopy.

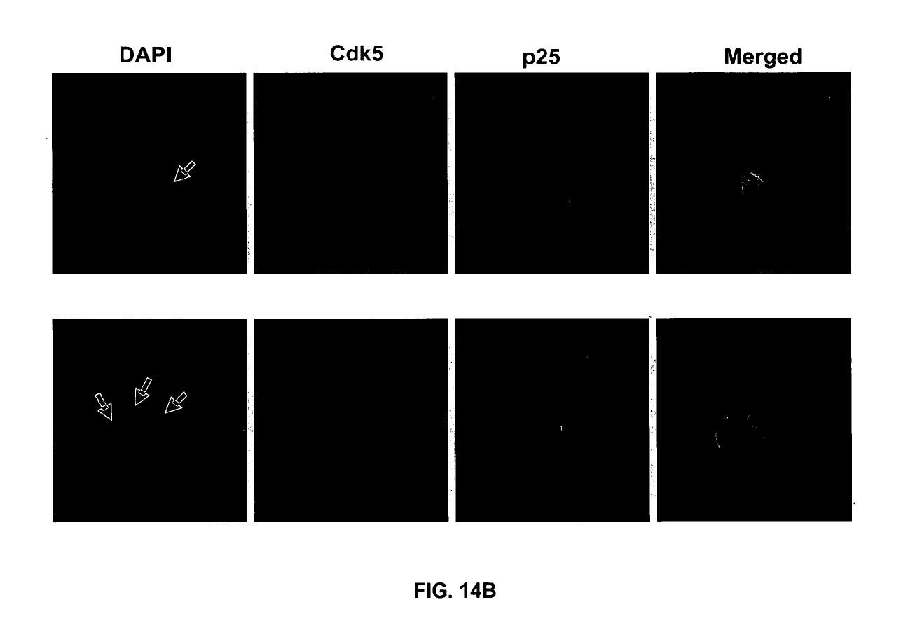

In addition to tangle formation (aggregation), it is known in the art that the tau protein in AD brain has undergone a type of chemical modification known as “phosphorylation”, which is believed to enhance tangle formation. Phosphorylation of tau is catalyzed by two enzymes known as CDK5 and GSK3, respectively, and over-expression of both enzymes in genetically-engineered mice leads to neurofibrillary tangle accumulation and neuronal cell death. Consequently, CDK5 and GSK3 appear to desirable drug targets, in addition to tau polypeptide itself, in the quest to find interventions of tangle formation. In light of this, it was determined that cinnamon extract can inhibit the activity of GSK3 in a living cell system. Remarkably, the extract can also directly inhibit CDK5. In this context, the data shown in FIG. 14A shows that the co-expression of CDK5 and p25 in COS-7 cells is toxic. Toxicity is seen as shrunken cell nuclei visualized by DAPI staining (red arrows). The corresponding cells express CDK5 (green fluorescent protein) and p25 (Cy3 red). Cells that do not express CDK5 or p25 have normal nuclei (DAPI). FIG. 14B then shows that a cinnamon extract prevents Cdk5/p25—induced toxicity. Cos-7 cells expressing CDK5 and p25 (red arrows) were treated with 0.05 mg/ml of cinnamon extract. Under these conditions, toxicity due to CDK5/p25 co-expression was not observed. Thus, the cinnamon extracts disclosed herein may act through multiple mechanisms to powerfully inhibit tau aggregation overall. Combined with the properties of non-toxicity, bio-availability and ease of production, this cinnamon extract is a highly favorable substance for development into an effective medicine to slow or prevent Alzheimer's disease.

The ability to inhibit CDK5 is of particular significance, because active CDK5 in the pancreas is essential for glucose desensitization, a hallmark of insulin resistance. Owing to its broad actions on insulin metabolism in the body and tangle formation in neurons, cinnamon extract appears to harness unique therapeutic potential for both AD and type II diabetes. In this context, embodiments of the invention also include methods of using the compositions disclosed herein to inhibit the phosphorylation of tau by cdk5/p25 kinase. One such embodiment of the invention comprises combining an amount of the proanthocyanidin compositions (e.g. via administration to a patient suffering from a tauopathy) with CDK5 in an amount sufficient to inhibits its phosphorylation of tau polypeptides. Optionally this method is practiced in an individual diagnosed with a tauopathy.

Certain embodiments of the invention comprise proanthocyanidin compositions characterized by one, two, three, four or more observed properties. In one embodiment the proanthocyanidin compositions are characterized by (a) common absorbance with tau at an isobestic point when tau is exposed to the proanthocyanidin; and/or (b) the capability of dissolving in water or aqueous solution; and/or (c) a decrease in absorbance spectra at about 279 nm and an increase at about 300 nm upon the addition of tau, wherein the change in absorbance at 300 nm represents ionization of a phenolic group; and/or (d) a dimer of the proanthocyanidin having a mass spectroscopy peak of 617 m/z, a trimer of the proanthocyanidin having a mass spectroscopy peak of 905 m/z, a tetramer of the proanthocyanidin having a mass spectroscopy peak of 1193 m/z, or having a pentamer of the proanthocyanidin having a mass spectroscopy peak of 1481 m/z; and/or (e) the capability of reducing the ability of tau to aggregate as measured by thioflavin T assays; and/or (f) the capability of reducing the ability of tau to aggregate as determine by SDS gel electrophoresis: and/or (g) the capability or reducing tau to aggregate as measured by light scattering; and/or (h) the capability of reducing the number and length and degree of tau filament formation as measured by electron microscopy studies. Typically, the proanthocyanidin composition includes monomers of catechin and/or epicatechin and derivatives of these such as dimers, trimers, tetramers, pentamers, as are known in the are and shown as FIG. 1 (see, also Dixon, R A et al. New Phytol. (2005) 165:9-28. Optionally, the proanthocyanidin composition affects insulin signaling. Optionally, the proanthocyanidin composition is capable of insulin-like biological activity.

Certain embodiments of the invention comprise a pharmaceutical composition containing one or more proanthocyanidin compounds. “Pharmaceutical composition” as used herein can comprise cinnamon extracts, proanthocyanidin compounds derived from other sources, or a mixture of cinnamon extracts and additional compounds that for example can stabilize the composition and/or aid in the reduction and/or inhibition of tau aggregates or the like.

In certain embodiments of the invention, additional compounds used in the art to treat tauopathies for example can be added to the pharmaceutical composition containing one or more proanthocyanidin compounds. For example, in the context of treatment of Alzheimer's disease, a patient may be benefited by administration of neurotransmission enhancing drugs, including anti-cholinesterase inhibitors such as, for example, tacrine, donepezil, rivastigamine, metrifonate, epastigimine, nicotine, pyridostigimine, neostigimine, physostigimine, ambenomium chloride and Gingko biloba; substances that increase brain catecholamines and/or reduce oxidative damage to neurons, including selegiline and vitamin E; non-steroidal drugs having anti-inflammatory properties, including statins (the statins also have immunosuppressant properties), such as lovastatin, simvastatin, pravastatin, fluvastatin, atorvastatin, rosuvastatin, and cerivastatin; aminoarylcarboxylic acid derivatives, such as enfenamic acid, etofenamate, flufenamic acid, isonixin, meclofenamic acid and tolfenamic acid; arylacetic acid derivatives, such as aceclofenac, acemetacin, bromfenac, clopirac, etodolac, fentiazac, indomethacin, oxametacine, and tropesin; arylbutyric acid derivatives, such as bumadizon, butibufen, fenbufen and xenbucin; arylcarboxylic acid derivatives, such as clidanac, ketorolac, and tinoridine; arylpropionic acid derivatives, such as alminoprofen, carprofen, fenoprofen, ibuprofen, indoprofen, ketoprofen, naproxen, pirprofen and zaltoprofen; pyrazoles, such as difenamizole and epirizole; pyrazolones, such as apazone, benzpiperylon, feprazone, oxyphenbutazone, pipebuzone, ramifenazone, and thiazolinobutazone; salicylic acid derivatives, such as acetaminosalol, aspirin, balsalazide, diflunisal, gentisic acid, imidazole salicylate, olsalazine, parsalmide, salicylsulfuric acid, sodium salicylate and sulfasalzine; and thiazinecarboxyamides, such as ampiroxicam, droxicam, isoxicam, lornoxicam, piroxicam and tenoxicam; and cholinergic agonists, including xanomeline, milameline, AF 102B and memric.

Additional embodiments of the invention comprise a method of evaluating the aggregated state of tau comprising: incubating tau in a first sample without a proanthocyanidin composition (which can be a preexisting and reusable control sample) and incubating tau in a second sample with a proanthocyanidin composition, and adding a thioflavin probe to the first sample and to the second sample at a time point, wherein the addition of the thioflavin probe causes a greater increase in fluorescence in the first sample without the proanthocyanidin composition as compared to the fluorescence of the second sample with the proanthocyanidin composition. Typically, the time point is 24 hours. In highly preferred embodiments, the aggregated state of tau is induced upon the addition of heparin or the like. Optionally, the proanthocyanidin composition comprises a water extract of cinnamomum zeylanicum, cinnamomum cassia, or cinnamomum loueirii.

Further embodiments of the invention provide methods of treating a mammal having a neurological condition or disorder, comprising administering to said mammal an effective amount of one or more proanthocyanidin compositions. Further embodiments of the invention include methods of diagnosing a patient with a neurological disorder or susceptible to a neurological disorder, comprising obtaining a sample from the patient and testing the sample for the presence of a tau aggregations using one or more proanthocyanidins as a probe for such aggregations.

Models for studying the development of, and/or pathologies associated with neurodegenerative diseases, and some agents that can alter such development and/or pathologies are known in the art for example methods to detect tauopathies are known in the art (see, e.g., U.S. Pat. Nos. 6,803,233, 6,781,029, 6,693,226, 6,680,173, 6,664,443, 6,563,015, 6,541,468, 6,479,528, 6,475,723, 6,444,870, 6,013,646, 6,680,173, 6,900,293, 6,232,437, 5,733,734, 6,500,674, 6,444,870, 5,994,084, 5,861,257, 6,717,031, 6,664,443, 6,563,015 6,475,723 6,221,670, 6,010,913, 6,037,521, and U.S. Patent Application No. 20060112437, the contents of each of which are herein incorporated by reference). Additional models for studying the development of, and/or pathologies associated with diabetes, and some agents that can alter such development and/or pathologies are known in the art for example mouse models to study diabetes are known in the art (see, e.g., U.S. Pat. No. 5,866,546, the contents of which are herein incorporated by reference).

In certain embodiments of the invention, animal models of neurological conditions or disorders including those noted above can be used to examine the effects of the proanthocyanidin compositions disclosed herein, as well as these agents in combination with each other and/or other therapeutic agents known in the art. In illustrative protocols for the experimental testing of one or more of the proanthocyanidin compositions disclosed herein, a number of age and gender matched animals from an animal model can be assigned to one of multiple test and/or control groups (e.g. JNPL3 transgenic mice as disclosed in Sahara et al., J Neurochem. 2005 September; 94(5):1254-63; and/or tau-P301L transgenic mice as disclosed in Terwel et al., J Biol Chem. 2005 Feb. 4; 280(5):3963-73). A first test group of these animals can then be administered a selected proanthocyanidin composition according to a specific administration protocol. Conditions for other test groups can be varied according to standard practices, for example: by administering a different dose of the proanthocyanidin compositions, by administering a different schedule of the proanthocyanidin compositions; by administering different proanthocyanidin compounds; by using a combination of agents (e.g. the proanthocyanidin compositions in combination with a cholinesterase inhibitor); by using a different route of administration (e.g. intravenous administration) etc. One or more groups of animals can serve as a control, for example one that receives sterile phosphate buffered saline according to the same course of administration as a test group that receives the proanthocyanidin.

At some period of time after receiving the proanthocyanidin composition, a test and a matched control group of these animals can then be compared for example to examine and/or characterize the effects of a proanthocyanidin composition in vivo. For example, samples comprising neuronal cells from a specific tissue or organ (e.g. the brain) from test and control groups of these animals can be evaluated by a technique such as magnetic resonance microscopy and/or immunohistochemical analysis in order to compare the status of neuronal cells in these groups (see, e.g. Petrik et al., Neuromolecular Med. 9(3):216-29 (2007)). Alternatively, samples obtained from these groups can be evaluated by a technique such as multi-photon microscopy in order to demonstrate phenomena such as altered neurite trajectory, dendritic spine loss or thinning of dendrites (see, e.g. Tsai et al., Nat. Neurosci. 7, 1181-1183 (2004): and Spires et al., J. Neurosci. 25, 7278-7287 (2005)). Alternatively, blood or other tissue samples obtained from these groups can be subjected to ELISA protocols designed to measure levels of markers of inflammation and/or apoptosis such as IL-1β, TNF-α, IL-10, p53 protein, interferon-γ, or NF-kappaB (see, e.g. Rakover et al., Neurodegener. Dis. 4(5):392-402 (2007); and Mogi et al., Neurosci Lett. 414(1):94-7 (2007)). Alternatively, animals from a test and a matched control group can be compared in behavioral test paradigms known in the art, for example the Morris water maze or object recognition tests (see, e.g., Hsiao et al., Science 274, 99-102 (1996); Janus et al., Nature 408, 979-982 (2000); Morgan et al., Nature 408, 982-985 (2000); and Ennaceur et al., Behav. Brain Res. 1988; 31:47-59). The results of comparisons between test and matched control groups of animals will allow those skilled in the art to examine the effects of proanthocyanidin compositions in vivo in the animal models.

Alzheimer's disease (AD) currently affects 5 million people in the U.S. and costs over 1 billion dollars annually in expense and lost productivity. The cause of Alzheimer's disease is not known, but the greatest risk factor is age. People over 65 yrs of age are at 3% risk, while over age 85 the risk of AD is >50%. Because the population of our seniors continues to grow disproportionately, it is predicted that 16 million people will have AD in the year 2050. If left unabated, the burden of AD on our health care system will be unmanageable. At present, there is no cure or preventative for AD. As noted herein, an extract of common cinnamon contains a class of small organic molecules that inhibit several key processes in AD. The extract itself exhibits potent inhibitory activity, is orally available, non-toxic, and the bio-active molecules are brain permeable. The extract is readily produced in large quantities, and can be encapsulated in powder form for oral administration.

Certain embodiments of the invention comprise administering a cinnamon extract comprising one or more proanthocyanidin compounds to a patient diagnosed with Alzheimer's disease in an amount effective to inhibit tau aggregation in vivo. Optionally, diagnosis of Alzheimer's disease in a patient may be based on the criteria of the Diagnostic and Statistical Manual of Mental disorders, 4th Edition (DSM-IV-TR) (see, e.g. American Psychiatric Association. Diagnostic and statistical manual of mental disorders, 4th Edition-text revised. Washington, D.C.: 2000). Briefly, the DSM-IV-TR criteria include: (A) the development of multiple cognitive deficits manifested by both memory impairment and one or more of the following: (1) aphasia; (2) apraxia; (3) agnosia; or (4) disturbances in executive functioning; (B) the cognitive deficits represent a decline from previous functioning and cause significant impairment in social or occupational functioning; (C) the course is characterized by gradual onset and continuing decline; (D) the cognitive deficits are not due to other central nervous system, systemic, or substance-induced conditions that cause progressive deficits in memory and cognition; and (E) the disturbance is not better accounted for by another psychiatric disorder. Alternative criteria by which diagnosis of Alzheimer's disease may be made include those based on the National Institute of Neurological and Communicative Disorders and Stroke-Alzheimer's Disease and Related Disorder Association (NINDS-ADRDA) working group criteria for Alzheimer's disease (see, e.g. McKhann et al., Neurology 1984; 34: 939-944). Briefly, the NINCDS-ADRDA criteria for possible Alzheimer's disease includes a dementia syndrome with an atypical onset, presentation, or progression and without a known etiology where any co-morbid diseases capable of producing dementia are not believed to be the cause. The NINCDS-ADRDA criteria for probable Alzheimer's disease includes dementia established by clinical and neuropsychological examination and involves (a) progressive deficits in two or more areas of cognition, including memory; (b) onset between the ages of 40 and 90 years; and (c) absence of systemic or other brain diseases capable of producing a dementia syndrome, including delirium. The NINCDS-ADRDA criteria for definite Alzheimer's disease includes meeting the criteria for probable Alzheimer's disease and has histopathologic evidence of Alzheimer's disease via autopsy or biopsy.

Revised NINDS-ADRDA diagnostic criteria have been proposed in Dubois et al., The Lancet Neurology, Volume 6, Issue 8, August 2007, Pages 734-746. As outlined briefly below, to meet this criteria for probable Alzheimer's disease, an affected individual must fulfill criterion A (the core clinical criterion) and at least one or more of the supportive biomarker criteria noted in B, C, D, or E. In this context, criterion A is characterized by the presence of an early and significant episodic memory impairment that includes the following features: (1) gradual and progressive change in memory function reported by patients or informants over more than 6 months; (2) objective evidence of significantly impaired episodic memory on testing: this generally consists of recall deficit that does not improve significantly, or does not normalize with cueing or recognition testing, and after effective encoding of information has been previously controlled; (3) the episodic memory impairment can be isolated or associated with other cognitive changes at the onset of AD or as AD advances. Criterion B is characterized by the presence of medial temporal lobe atrophy, as shown for example by: volume loss of hippocampi, entorhinal cortex, amygdala evidenced on MRI with qualitative ratings using visual scoring (referenced to well characterized population with age norms) or quantitative volumetry of regions of interest (referenced to well characterized population with age norms). Criterion C is characterized by an abnormal cerebrospinal fluid biomarker, for example low amyloid β₁₋₄₂ concentrations, increased total tau concentrations, or increased phospho-tau concentrations, or combinations of the three. Criterion C is characterized by a specific pattern on functional neuroimaging with PET, for example reduced glucose metabolism in bilateral temporal parietal regions. Criterion E is characterized by proven AD autosomal dominant mutation within the immediate family. AD is considered deftine if the following are present: (1) both clinical and histopathological (brain biopsy or autopsy) evidence of the disease, as required by the NIA-Reagan criteria for the post-mortem diagnosis of AD; criteria must be present (see, e.g. Neurobiol Aging 1997; 18: S1-S2); and (2) both clinical and genetic evidence (mutation on chromosome 1, 14, or 21) of AD; criteria must be present.

In certain embodiments of the invention, the effect of cinnamon extract comprising one or more proanthocyanidins disclosed herein on neurological disorders such as Alzheimer's disease (AD) in humans can be examined, for example, through the use of a cognitive outcome measure in conjunction with a global assessment (see, e.g. Leber P: Guidelines for the Clinical Evaluation of Antidementia Drugs, 1st draft, Rockville, Md., US Food and Drug Administration, 1990). The effects on neurological disorders, such as AD, can be examined for instance using single or multiple sets of criteria. For example, the European Medicine Evaluation Agency (EMEA) introduced a definition of responders corresponding to a prespecified degree of improvement in cognition and stabilization in both functional and global activities (see, e.g. European Medicine Evaluation Agency (EMEA): Note for Guidelines on Medicinal Products in the Treatment of Alzheimer's Disease. London, EMEA, 1997). A number of specific established tests that can be used alone or in combination to evaluate a patient's responsiveness to an agent are known in the art (see, e.g. Van Dyke et al., AM J Geriatr. Psychiatry 14:5 (2006). For example, responsiveness to an agent can be evaluated using the Severe Impairment Battery (SIB), a test used to measure cognitive change in patients with more severe AD (see, e.g. Schmitt et al., Alzheimer Dis Assoc Disord 1997; 11(suppl 2):51-56). Responsiveness to an agent can also be measured using the 19-item Alzheimer's Disease Cooperative Study-Activities of Daily Living inventory (ADCSADL19), a 19-item inventory that measures the level of independence in performing activities of daily living, designed and validated for later stages of dementia (see, e.g. Galasko et al., J Int Neuropsychol Soc 2005; 11:446-453). Responsiveness to an agent can also be measured using the Clinician's Interview-Based Impression of Change Plus Caregiver Input (CIBIC-Plus), a seven-point global change rating based on structured interviews with both patient and caregiver (see, e.g. Schneider et al., Alzheimer Dis Assoc Disord 1997; 11(suppl 2):22-32). Responsiveness to an agent can also be measured using the Neuropsychiatric Inventory (NPI), which assesses the frequency and severity of 12 behavioral symptoms based on a caregiver interview (see, e.g. Cummings et al., Neurology 1994; 44:2308-2314).

Oral preparations for example containing proanthocyanidin can be formulated according to known methods for preparing pharmaceutical compositions. In general, the proanthocyanidin therapeutic compositions are formulated such that an effective amount of the proanthocyanidin is combined with a suitable additive, carrier and/or excipient in order to facilitate effective oral administration of the composition. For example, tablets and capsules containing proanthocyanidin can be prepared by combining (e.g., concentrated or lyophilized proanthocyanidin) with additives such as pharmaceutically acceptable carriers (e.g., lactose, corn starch, microcrystalline cellulose, sucrose, maltitol, glycerol, propylene glycol, Tris, etc.), binders (e.g., alpha-form starch, methylcellulose, carboxymethylcellulose, hydroxypropylcellulose, hydroxypropylmethylcellulose, polyvinylpyrrolidone), disintegrating agents (e.g., carboxymethylcellulose calcium, starch, low substituted hydroxy-propylcellulose), surfactants (e.g., Tween 80, polyoxyethylene-polyoxypropylene copolymer), antioxidants (e.g., L-cysteine, sodium sulfite, sodium ascorbate), lubricants (e.g., magnesium stearate, talc), amino acids (e.g. leucine, alanine, histidine, etc.) or the like.

Further, the proanthocyanidins of the present invention can be mixed with a solid, pulverulent or other carrier, for example lactose, saccharose, sorbitol, mannitol, starch, such as potato starch, corn starch, millopectine, cellulose derivative or gelatine, and can also include lubricants, such as magnesium or calcium stearate, or polyethylene glycol waxes compressed to the formation of tablets. By using several layers of the carrier or diluent, tablets operating with slow release and/or targeted release can be prepared. Liquid preparations for oral administration can be made in the form of elixirs, syrups or suspensions, for example and a mixture of sugar, ethanol, water, glycerol, propylene, glycol, amino acid, salt and possibly other additives of a conventional nature.

The following sections describe methods for isolating the aforementioned compounds. Methods of isolating are known in the art (see, e.g., U.S. Pat. Nos. 6,926,914, 6,800,433, 6,720,353, 6,608,102 6,429,202, 6,426,080, 6,395,280, 6,291,517, 6,274,179, 6,264,997, 6,245,336, 6,228,387, 6,210,681, 6,210,681, 5,804,597, 5,773,262, 5,650,432, 5,494,661, 5,211,944, 4,797,421, 6,200,259, 6,200,569, 6,500,469, and 6,495,593, the contents of each of which are herein incorporated by reference). These methods can be used for example to isolate polyphenolic polymers found in cinnamon that potentiate insulin action, and can be beneficial in the control of glucose intolerance and diabetes.

Additionally, methods for stabilizing proanthocyanidins, for example, are known in the art. It is known that this substance can be unstable in the presence of oxygen, for example oxidative polymerization etc. rapidly occurs in the presence of oxygen, which discolors the proanthocyanidin. One method for preventing oxidative polymerization utilizes (and the composition contains) proanthocyanidin, and an amino acid having a hydroxyl group or a dipeptide containing said amino acid (see, e.g., U.S. Pat. Nos. 6,685,970 and 6,506,419, the contents of each of which are herein incorporated by reference). The production of proanthocyanidins for example can be increased for scale-up by contacting the proanthocyanidin-containing solution with tanners or the like in the extraction of the proanthocyanidins (see, e.g., U.S. Pat. No. 5,814,494, the contents of which are herein incorporated by reference).

Proanthocyanidins can be obtained by extracting and purifying these compounds from various plants such as cinnamon, grapes, apples, barley, persimmons, coconuts, cacao, pines, blueberries, strawberries, adzuki beans or peanuts, for example. Suitable parts of a plant such as fruits, seeds, leaves, stems, roots or rootstocks as the starting material are collected at an appropriate season and directly used as the extraction material, or more preferably after first subjecting the collected plant parts to a drying step such as air-drying. Such plants preferably belong to the genera Cinnamomum, Vitis, Malus, Hordeum, Diospyros, Cocos, Theobroma, Pinus, Vaccinium, Fragaria, Phaseolus or Arachis. Proanthocyanidin can also be obtained optionally by purification from fermentation products of suitable extracts, such as a wine, an apple wine or a beer. Proanthocyanidin-containing plants can be members of the Coniferiae class including plants from the order Coniferales and particularly from the family Pinaceae (including pines); members of the family Filices (including palms); monocot plants form the order Arecales, including members of the families Pandanales, Arales, Najadales, Restionales, Poales (including grains such as sorghum, barley and others), Juncalaes, Cyperales (including cypress), Typhales, Zingiverales, and Lihales (including lilies); dicot plants from the orders Laurales (including laurel, cinnamon), Fagales (including oak), Casuarinales, Dilleniales, Malviales (including cotton), Salicales, Ericales (including cranberries, blueberries, rhododendron), Ebenales, Rosales (including roses, blackberries and other related berries, apples, peaches, plums), Fabales (including legumes, wysteria), Myrtales, Proteales, Rhamanales (including grapes) and Sapindales. The preferred plants can be dicots Ericaceae, which includes the Vaccinium spp., Rosaceae and Vitaceae, which includes the Vitis spp.; and the conifers of the Pinaceae family. The Vaccinium spp. include, but are not limited to, plants with cranberry-type berries such as V. macrocarpon (cranberry), V. vitis-idaea (mountain cranberry, cow berry, lingonberry) and V. oxycoccus (European cranberry); and plants with blueberry fruit such as V. augustifolium (low sweet blueberry), V. ashei (Rabbiteye blueberry), V. corymbosum (high bush blueberry), V. lamarckii (early sweet blueberry) and V. myrtillus (bilberry, European blueberry). The Vitis spp. include, but are not limited to, V. labrusca (Fox grape), V. rotunddifolia (muscadine, scuppenong), V. vinifera (European grape) and all interspecifc hybrids with other Vitis species (see, e.g., U.S. Pat. No. 6,608,102, the contents of which are herein incorporated by reference).

Extraction of proanthocyanidin from the collected extraction material can be carried out from finely ground powder or quills in the case of cinnamon. As the extraction solvent, one or more hydrophilic or lipophilic solvents can be used alone, sequentially, or together in admixture. Such solvents are preferably selected from solvents such as water; alcohols such as ethanol, methanol or isopropanol; ketones such as acetone and methyl ethyl ketone; and esters such as methyl acetate and ethyl acetate. The extraction temperature is generally from 0 to 100° C. with water. The water can be distilled or nanopure for example.

After extraction, the composition can be further purified using HPLC (High Performance Liquid Chromatography) for scale-up or production of the pharmaceutical composition for example as is known in the art. Methods to characterize and/or study the function of compositions such as proanthocyanidins, for example NMR, mass spectrometry, HPLC, spectrometry studies, fluorescence and the like are known in the art.

Illustrative proanthocyanidins of the invention include proanthocyanidin polymers having linear chains of 5,7,3′,4′ tetrahydroxy or 5,7,3′,4′,5′ pentahydroxy flavonoid 3-ol units linked together through common C(4)-(6) and/or C(4)-C(8) bonds. These are typically the type B linked polyphenols. In addition some monomeric units can be linked by a C(2)-O—C(7) bond. These are typically type A linked polyphenols. Biosynthetic studies have indicated that proanthocyanidin polymers can consist of monomer units that are generally termed “leucoanthocyanidin” of the polymer chain may be based on either of two stereochemistries of the C-ring, at the 2 and/or 4 position designated cis (called epicatechins) or trans (called catechin). Therefore, the polymer chains can be based on different structural units, which create a wide variation of polymeric proanthocyanidins and a large number of possible isomers C13 NMR has been useful to identify the structures of polymeric proanthocyanidins and recent work has elucidated the chemistry of di-, tri- and tetra-meric proanthocyanidins. Larger polymers of the flavonoid 3-ol units are predominant in most plants, and are found with average molecular weights above 2,000 daltons, containing 6 or more units (see, e.g., U.S. Pat. No. 5,494,661, the contents of which are herein incorporated by reference).

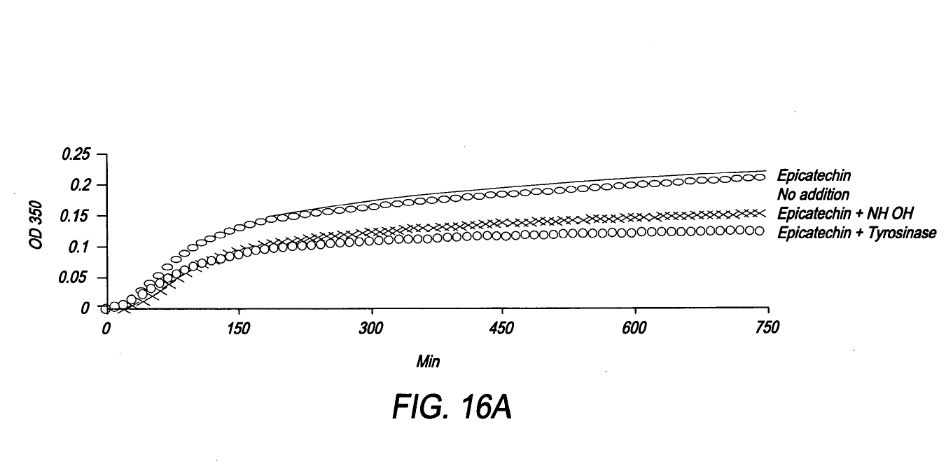

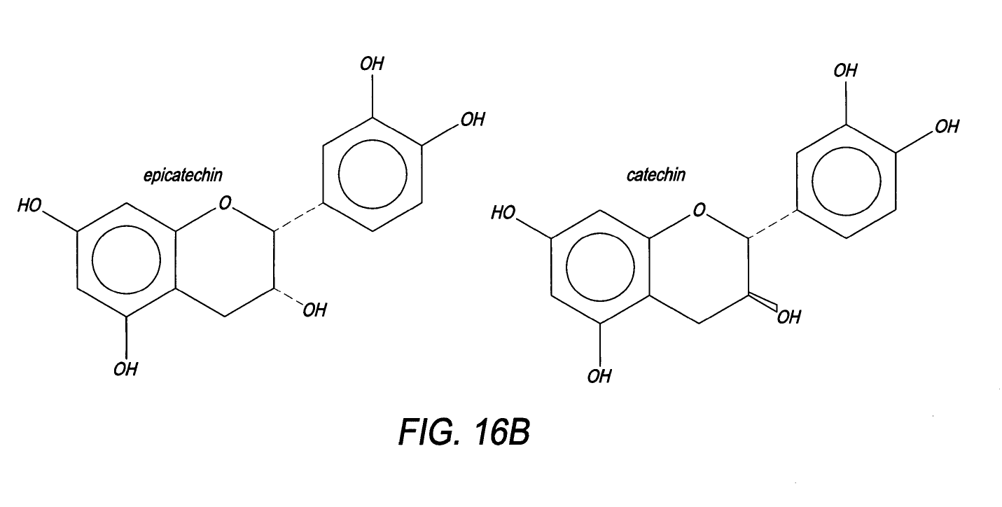

The disclosure provided herein provides evidence that monomeric epicatechins and catechins can also inhibit tau aggregation. Monomeric forms of catechins and epicatechins and their oligomeric products (i.e. proanthocyanidins) are found in varying relative amounts in various species of cinnamon, namely Cinnamomum zeylancium, Cinnamomum cassia, and Cinnamomum loureiroi. Unlike proanthocyanidin A-type trimer described herein, epicatechin does not have intrinsic tau aggregation inhibitory activity on its own. However, a treatment regimen for epicatechin such as a treatment with ammonia (pH 9-9.5) overnight (18 hrs) at room temperature (23° C.) causes an increase in its insulin-like activity, and furthermore causes it to become effective in inhibiting tau aggregation. Similar effects are observed upon the enzymatic oxidation of epicatechin by tyrosinase enzyme. Because catechins and epicatechins are known to be powerful antioxidants, it is likely that in the presence of reactive oxygen free-radical species in the brain generated by conditions of natural oxidative stress, these compounds may become oxidized—and activated—as inhibitors of tau aggregation in neuronal cells, possibly resulting in inhibition of neurodegeneration. Epicatechin is absorbed and metabolized after oral ingestion and is found to be brain-permeable like other catechins from green tea, and it is believed that their brain permeability is greater than that of corresponding proanthocyanidin oligomers including the A-type trimer. In this context, the data presented in FIG. 16A shows that the oxidation of epicatechin monomer results in tau aggregation inhibitory activity in vitro. Thus epicatechin and catechin may constitute a significant component of the protective effect of cinnamon extract against tau aggregation in humans. For methods and materials relating to this disclosure see, e.g. Manal et al, (2002), Free Radical Biology and Medicine, 33, 1693-1702; and Mandel et al., (2006), Molecular Nutrition and Research, 50, 229-234.

As disclosed herein, certain embodiments of the invention use the proanthocyanidins of the invention as probes to identify and/or characterize tau in a variety of contexts. In this context, the use of probes for example such as spectroscopic probes to study protein aggregation is well known in the art. One example of a small molecular spectroscopic probe is Thioflavin-T. Thioflavin-T is a fluorescent dye that has been widely used for the detection of amyloid fibrils for example. Recently, Thioflavin-T has been used to elucidate the mechanism of fibril formation in insulin. In the presence of fibrils, and perhaps other protein configurations as well, Thioflavin-T gives rise to a new excitation maximum at about 450 nm and enhanced emission at about 482 nm when bound to a fibril protein form. Unbound Thioflavin-T is essentially non-fluorescent at these wavelengths. Other small molecules can be used as probes of the changes in protein structure from native to non-native states, such as aggregated states. Examples of other small molecular, spectroscopic probes are the “exposed hydrophobic patch” probe and the “exposed coordination site” probe. As is the case with Thioflavin-T, these small molecular, spectroscopic probes yield a spectroscopic change upon binding to a non-native or aggregated form of the protein of interest, such as a change in fluorescence, a change in absorbance, a change in circular dichroism, and the like. The “hydrophobic patch” probe preferentially binds to exposed hydrophobic patches of a protein. These hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline, or the like. Other spectroscopic probes are metal-amino acid complexes, such as cobalt metal complexes of hydrophobic amino acids, such as phenylalanine, leucine, isoleucine, methionine, and valine, or the like (see, e.g., U.S. Pat. No. 6,737,401, the contents of which are herein incorporated by reference).

Mass spectrometry for example can be used to characterize compounds and to establish the structure of a new compound for example. The mass spectrum can help establish the structure of a new compound in several different ways: it can give an exact molecular weight; it can give a molecular formula, and can indicate the presence of a molecule of certain structural units. Mass spectroscopy for example can be used to show that the water extract and the reconstituted sample both comprise proanthocyanidin, as evidenced by same/similar mass spectroscopy peaks and to show that the method disclosed herein to prepare the solids from the extract does not destroy the proanthocyanidin.

Light scattering studies can be conducted to determine the aggregated state of proteins for example as is known in the art. A decrease in light scattering as measured by a reduction in absorbance spectra can show an increase in the disaggregated state of the protein, for example. In these studies, plots of time vs. absorbance at a particular wavelength can be determined.

As is known in the art, spectroscopy studies can be conducted to determine whether two species bind together, for example. A plot of wavelength vs. absorbance can be determined, and an isosbestic point can be determined. An isobestic (or isosbestic) point is a point on a isobestic plot. This point represents a wavelength at which the absorption spectra of two species cross each other. The appearance of an isobestic point during a chemical reaction can be evidence that there are only two species present at a constant total concentration. For example, it can be shown that two species bind together when the addition of a second species to a first species causes a decrease in the absorbance and when an isobestic point appears on the plot after the addition of the second species, wherein the appearance of the isobestic point shows that the two species have a common absorbance at a certain wavelength and therefore bind to each other (see, e.g., Nakae Y. et al, J Histochem Cytochem 1997 October. 45(10):1417-1425 and Wohlrab F., Acta Histochem 1998 84(2): 187-194, the contents of each of which are herein incorporated by reference).

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### EXAMPLES

The Examples below provide illustrative methods and materials that can be used in the practice of the invention.

### Example 1

### Typical Methods and Materials used in the Practice of the Invention

### Preparation of Water Extracts and Reconstituted Compounds

Water extracts of cinnamon are easy to prepare and purification of proanthocyanidins, if desired, can be done by HPLC chromatography of the extracts. Mass spectrometric methods for characterization of the purified proanthocyanidins and means to study their effects on tau aggregation are described in scientific journals

In a first illustrative embodiment of an extraction procedure of the invention, an extract is obtained by using nanopure water equal to 10 times the weight of cinnamon. The mixture is heated for 10 minutes at 95° C., cooled, and centrifuged at 14,000 rpm. The nearly clear supernatant is centrifuged again and then the clear supernatant is filtered through 0.4 and 0.2 micron filters and lyophilized. A fluffy pale tan colored powder can be obtained. The solids are then dissolved in water (10 mg/ml) and mass spectrometry of the solution shows the presence of the proanthocyanidins found in the initial extract. Extracts prepared from Cinnamomum cassia or Cinnamomum loueirii (containing B linked proanthocyanidin) or from Cinnamomum zeylanicum (containing A linked proanthocyanidin) all inhibit with near equal potency tau aggregation.

In a second illustrative embodiment of an extraction procedure of the invention 5 grams of finely ground Cinnamomum zeylanicum is added to 50 mL of 40° C. nanopure H2O in a round bottom flask under constant stirring and incubated for 10 min at 40° C. The entire reaction is then removed and centrifuged for 10 minutes at 5,000 rpm in a SS-34 rotor at 4° C. The supernatant is then removed and brought to 1° C. with constant stirring in an ice bath for 30 minutes. The resultant sample was then removed and centrifuged at 15,000 rpm for 30 minutes at 4° C. in an SS-34 rotor. The resultant supernatant is then removed and passed through a 0.22 μm filter, flash frozen in liquid nitrogen and lyophilized overnight to obtain a soft, tan powder.

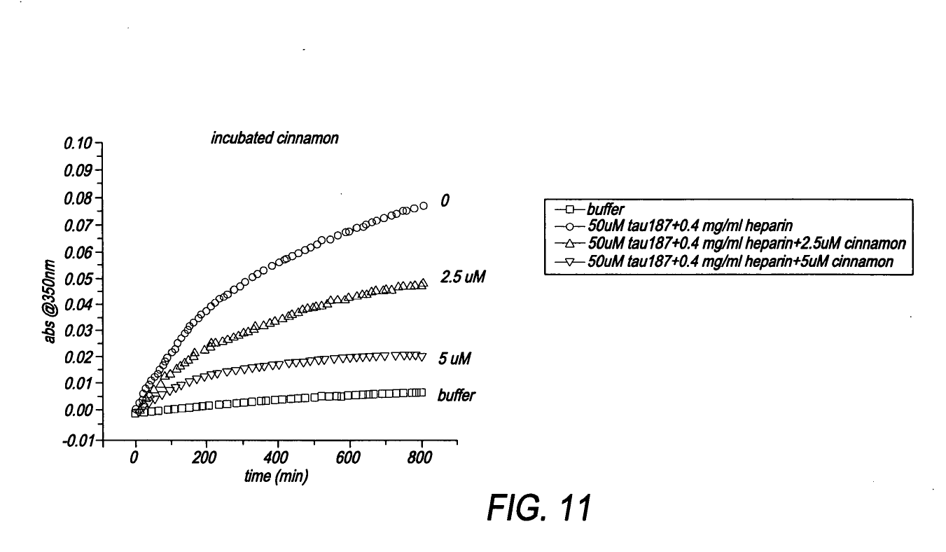

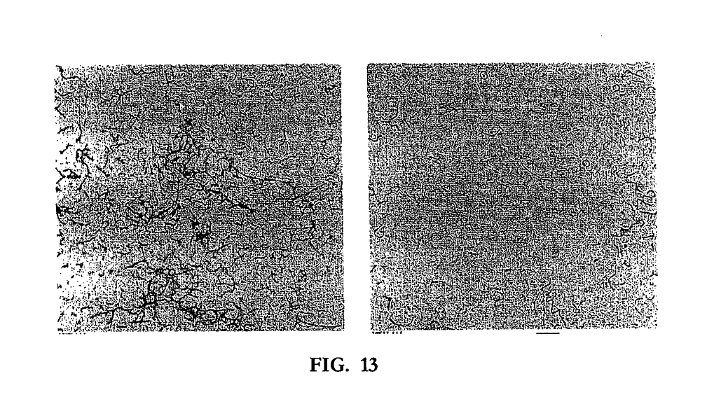

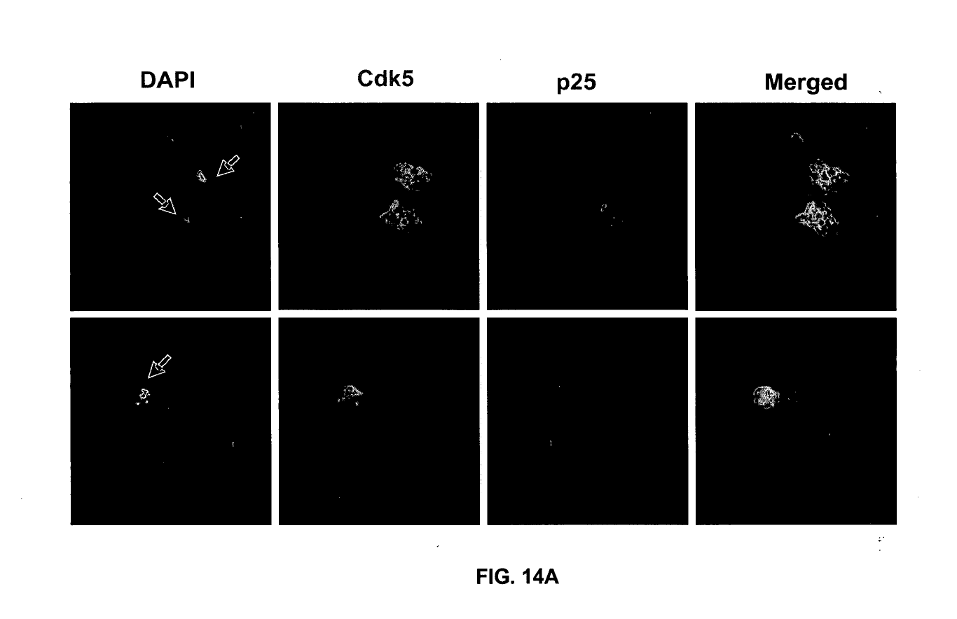

As shown for example in FIGS. 11, 12, 13 and 16 in certain embodiments of the invention, the oxidation state of a component of a proanthocyanidin and/or the associated constituents in the cinnamon extracts disclosed herein can be further manipulated (e.g. oxidized or reduced) as part of the preparation process, for example by exposing them to one of the variety of oxidizing or reducing agents known in the art. Consequently, in certain embodiments of the invention, the proanthocyanidins and/or the associated constituents in the cinnamon extracts are further prepared to place them in, for example, an oxidized or non-oxidized forms. In this context, illustrative references that deal with mass spectroscopy of cinnamon extracts and their oxidation include for example Pavlovich et al., (2005), “Electrospray MS Profiling of Proanthocyanidin Oligomers in Commercially Available Varieties of Cinnamon and Cassia”, 53rd ASMS Conference on Mass Spectrometry and Allied Topics, San Antonio, Tex., June 5-9, ThP 318; and Lampke et al., Activation of Insulin-like Activity of Proanthocyanidins from Cinnamon”, FASEB Meeting, San Francisco, CA, Abs. 612.2, the contents of which are incorporated by reference.

### Thioflavin Assay

In one illustrative embodiment of the invention, the effects of cinnamon proanthocyanidin or its extract can be determined by fluorescence measurements in the presence of a thioflavin derivative. If aggregation proceeds through formation of an ordered structure, enhancement of thioflavin occurs. This method can be used to quantify the effects of the proanthocyanidins on the rate and extent of tau aggregation.

An alternative method is to incubate tau in the absence of thioflavin under conditions that cause aggregation in the absence and presence of cinnamon proanthocyanidin. At different times aliquots are removed to measure the aggregation by examining the fluorescence of thioflavin T added to these aliquots. As thioflavin can affect aggregation itself and could compete partly with binding of proanthocyanidin this modified assay could maximize the effect of proanthocyanidin. Because it would necessitate removing aliquots from the reaction at different times and mixing in new reagents, it is less convenient and prone to additional errors.

Water extracts of cinnamomum loureirii, cassia, and zyelanicum enriched in proanthocyanidins inhibit aggregation of a tau fragment (e.g. tau 187) containing the structural features of tau necessary for aggregation. This conclusion was reached by measuring changes in fluorescence of thioflavin T under conditions well documented to cause formation of tau aggregates. Additionally, purified proanthocyanidin A reduces aggregation of tau as measured by changes of thioflavin T fluorescence.

TABLE 1
Thioflavin T fluorescence. Fluorescence was measured
after 24 h aggregation at 30 C. The samples are
the same of those in FIG. 11. I added 25 uM thioflavin
T at the moment fluorescence was checked.

| | t 0 | t 24 hrs |
|---|---|---|
| | (cps/mV) | (cps/mV) |
| Buffer | 2.5 × 10⁶ | 1.5 × 10⁶ |
| Tau + hep | 2.5 × 10⁶ | 10 × 10⁶ |
| Tau + hep + | 2.0 × 10⁶ | 6 × 10⁶ |
| 2.5 uM cinnamon | | |
| Tau + hep + | 2.0 × 10⁶ | 4.5 × 10⁶ |
| 10 uM cinnamon | | |

inhibition by fluorescence measurements

### Light Scattering Assay

In one illustrative embodiment of the invention, the effects of cinnamon extract or purified cinnamon proanthocyanidins on tau aggregation can be followed readily in a spectrophotometer by measuring the change in absorbance at 350 nm. The association of tau promoted by heparin causes an increase in absorbance. Cinnamon compounds inhibit this response and information can be obtained about the processes involved by comparing the rates and extent of change with and without cinnamon compounds. Different buffers and temperatures may be used if desired to study factor influencing aggregation.

Light scattering measurements show that water extracts of Cinnamomum zeylanicum, Cinnamomum cassia, Cinnamomum loueirii, cinnamon proanthocyanidin A or cinnamon proanthocyanidin B2 inhibits the aggregation of tau polypeptides.

Spectrophotometric measurements show that proanthocyanidin A binds directly to tau by the changes seen in its absorption spectrum.

### Electron Microscopy

In one illustrative embodiment of the invention, transmission electron microscopy, is a highly desirable technique for studies of aggregation with heparin and water or with heparin and purified proanthocyanidin or cinnamon extracts from Cinnamomum zeylanicum, Cinnamomum cassia, or Cinnamomum loueirii. It provides definition of what the aggregates actually are, e.g., filaments and their abundance, sizes, and shapes.

Transmission electron microscopy shows that formation of tau filaments is inhibited by cinnamon proanthocyanidin A or water extracts from Cinnamon zyelanicum, Cinnamon cassia, or Cinnamomum loueirii under aggregating conditions.

### Gel Electrophoresis

In one illustrative embodiment of the invention, gel electrophoresis also can be used to study effects on aggregation. The advantage here is that simple equipment is needed for these measurements but the system provides less definition of the effects of the inhibitors than electron microscopy. Full-length tau or tau 187 (50 uM) is induced to undergo aggregation with heparin (0.1 mg/ml) for 16 hours at room temperature in the presence of varying concentrations of cinnamon extract or cinnamon proanthocyanidin type A trimer. Samples are then treated with 1% sarkosyl for 1 hr, centrifuged (105,000 g×1 hr), and the pellets (P) versus supernatants (S) then analyzed by SDS-PAGE. The increase in the ratio of tau in the pellet compared to the supernatant is a measure of aggregation and cinnamon extract or purified proanthocyanidin is found to decrease this ratio.

Gel electrophoresis results show that less aggregation of tau occurs in the presence of water extracts of Cinnamon zeylanicum, Cinnamomum cassia, Cinnamomum loueirii or cinnamon proanthocyanidin A.

### Rodent Models

Rodents such as mice, rats, and hamsters have been used for models for type 2 diabetes and neurodegenerative processes in illustrative embodiments of the invention. In some studies, results show that impaired insulin action (insulin resistance) can promote tau phosphorylation. Experiment protocols with Wistar rats fed a high fructose diet that has been shown to induce resistance are in progress. Changes in the insulin signaling pathway and tau phosphorylation will be influenced by the diet and these effects will be altered by whole cinnamon or its water soluble extract. Some effects, e.g., protein phosphorylation can be studied by using immunohistochemistry or the like. Specific strains on mice may be used for studies involving tau aggregation and formation of neurofilaments. One such strain is the App/RK strain of mice (see, e.g. Moechars et al., Neuroscience 91(3): 819-830 (1999)). Other transgenic murine lines include the APP23 and JNPL3 transgenic lines that express mutant Alzheimer's associated polypeptides and further exhibit neuronal cell loss. APP23 and JNPL3 transgenic mice thus provide models of Alzheimer's disease (see, e.g. McGowan et al., TRENDS in Genetics Vol. 22 No. 5 (2006).

In such studies, cinnamon products that display insulin potentiating activity or the like can be used. These products can be purified using HPLC for example and can be used to determine for example their effects on changes in tau. These cinnamon products or extract can be given to rodents orally or incorporated directly in the chow they eat. Long term feeding of mice, up to 9 months, with cinnamon extract will provide information about any toxicity associated with the daily intake of cinnamon compounds.

### Further Methods

Additional illustrative methods of the invention comprise additional methods such as microarray, tissue microarray, in vitro binding assays, Western Blots, Northern Blots, in situ hybridization, RNAi, or any assay known in the art for determination of protein signaling (such as insulin signaling) for example or for determination tau gene expression, protein aggregation, filament formation, or fibril formation or the like.

### Example 2

### Cinnamon Extract as a Potential Therapeutic for Alzheimer's Disease—Charaterizations of Effective Dosages

Proanthocyanidin polyphenolic molecules endogenous to ordinary cinnamon have been shown to be effective insulin mimetics in fat cells in vitro, and whole ground cinnamon has been shown to exhibit beneficial effects on diabetes in humans. In this context, the disclosure provided herein identifies a potential therapy for Alzheimer's disease based on an extract derived from cinnamon a plant material which is known to be safely tolerated by humans. As in the case with diabetes, certain active components (i.e. inhibitors of tau aggregation) are proanthocyanidin molecules. An effective dosage of cinnamon extract to administer to humans to potentially treat Alzheimer's disease is chracterized below based on known dosages effective in mimicking insulin signaling in vitro and to treat diabetes in humans.

TABLE 2
DOSE CHARACTERIZATIONS

| | | |
|---|---|---|
| Diabetes | | Diabetes |
| In Vitro studies | | Effective Dosage in |
| 0.1 mg/ml PA trimer | | Humans |
| stimulates insulin signaling in | | 1 gm/day whole |
| vitro.¹→ | | cinnamon |
| | | lowers blood |
| | | glucose, lipids, |
| | | cholesterol and |
| | | LDL in humans². |
| Alzheimer's disease | Considerations for dosing CE | Alzheimer's |
| In vitro studies→ | in humans: | disease |
| 0.17 mg/ml PA trimer | CE powder, which contains the | Estimated dosage |
| completely inhibits tau | PA trimer, is 7% (w/w) of whole | for humans |
| aggregation in vitro³. | cinnamon⁴. Therefore PA trimer | 1.7 gm/day |
| This is the technology | is enriched ~14x. | whole cinnamon |
| developed by Lew and Graves | CE is ~2x more potent than | /14 = 0.121 |
| at UCSB. | equivalent amount of PA trimer | gm/day CE |
| | alone⁵. | /2 = 0.06 |
| | Permeability of polyphenol | gm/day CE |
| | molecules to brain in rats is 10- | × 10 = 0.6 |
| | 20% that of other tissues⁶. | gm/day CE |
| | (Assume 10%) | ~600 mg/day (2-3 |
| | | capsules) |

Abbreviations: CE—cinnamon extract; PA—proanthocyanidin; AD—Alzheimer's disease
Footnotes
¹0.1 mg/ml PA trimer maximally stimulates insulin signaling in vitro. See, e.g. Graves et al. J. Am. College Nutr. (2001) 327-336 (See FIGS. 1 & 2, p330). FIG. 1 in this publication shows that 0.1 mg/ml of PA trimer (MHCP) causes maximal activation of the insulin receptor in vitro (i.e. in adipocyte cells). Insulin receptor activation is measured by the extent of a chemical modification to the receptor itself called “tyrosine phosphorylation”. Size and darkness of the bands in the figure reflect extent of tyrosine phosphorylation. FIG. 2 in this publication shows that the same concentration of PA trimer as in FIG. 1 causes a corresponding increase in glucose uptake, similar to that seen using physiological concentrations of insulin.
²1 gm whole cinnamon per day effectively lowers blood glucose and lipids in humans. See, e.g. Anderson et al. Diabetes Care (2003) 3215-3218 (See Tables 1-4). This study shows that physiological effects on several diabetic parameters in humans are brought about by administering 1 gm of whole ground cinnamon per day in comparison to 0.1 mg/ml of PA trimer in vitro (Graves et al. J. Am. College Nutr. (2001) 327-336) Increasing the dosage of whole cinnamon does not seem to increase efficacy in humans.
³0.17 mg/ml of PA trimer completely inhibits tau aggregation/AD fiber formation in vitro. Our studies show that, in vitro, concentrations of PA trimer (0.17 mg/ml) similar to those that activate insulin signaling (0.10 mg/ml, Graves et al. J. Am. College Nutr. (2001) 327-336) also inhibit tau aggregation.
⁴Our studies show that the PA trimer and other molecules are extracted from whole ground cinnamon with water, and the extract is freeze dried. The total mass of the freeze-dried powder (CE) is routinely found to be ~7% that of whole cinnamon. The PA trimer is therefore assumed to be ~14X enriched in CE compared to whole cinnamon.
⁵Our studies show that the concentration of PA trimer in CE is determined by HPLC (high performance liquid chromatography) in our laboratory. When the same amount of PA trimer in purified form is tested, it is found to be only ~½ as effective as CE in inhibiting tau aggregation. Thus CE has 2X the potency as PA trimer alone.
⁶See, e.g. Bishop et al. Brain Res. (1999) 358-366 (See p360, Table 1) These data show that in adult rats, brain represents 0.5-0.9% of the total body weight. Therefore, if a pharmacological agent were freely accessible to all body tissues, a maximum of 0.5-0.9% would be expected to accumulate in brain.

See, e.g. Weaver et al. FASEB J. (2007) 21 837.11

These researchers studied the tissue distribution of proanthocyanidin polyphenols similar or identical to those found in cinnamon, and showed that approximately 0.1% of total radiolabeled proanthocyanidin polyphenols administered orally to rats accumulates in brain. Since the maximum expected is 0.5-0.9%, then the permeability of these molecules to brain can be assumed to be 10-20% that of other tissues. Thus the dose of cinnamon extract for targeting brain processes should be 5-10× greater than for targeting other tissue.

The data included therein and the associated characterization of this data evidences that the proanthocyanidin compositions disclosed herein will for example, bind tau polypeptides and inhibit their aggregation in vivo. In particular, the disclosure presented herein teaches for example that: (1) the cinnamon proanthocyanidin compositions disclosed herein (and specific constituents of these components such as the proanthocyanidin A-type trimer) can bind to tau polypeptides, can inhibit tau polypeptide aggregation and can protect soluble tau polypeptides from aggregation in vitro in accordance with known scientific mechanisms and principles (see, e.g. the data disclosed in the figures); (2) such cinnamon proanthocyanidin compositions disclosed herein are safe and well tolerated in humans (see, e.g. U.S. Pat. No. 6,200,569); and (3) proanthocyanidins administered orally to mammals accumulates in brain, the specific organ where the tau aggregates are observed. In view of Applicants' findings and disclosure, one of skill in this art will reasonably expect the disclosed proanthocyanidin compositions to bind tau polypeptides and inhibit tau polypeptide aggregation in vivo. For this reason, the skilled artisan will reasonably expect animal models such as those noted above and the associated techniques for examining the various pathological processes observed these animal models to confirm the biological activity of proanthocyanidin compositions, as described herein.

TABLE 3
POLYPEPTIDE SEQUENCES

Tau-A (UniProtKB/Swiss-prot Isoform ID: P10636-3)
352 Amino Acids
MAEPRQEFEVMEDHAGTYGLGDRKDQGGYTMHQDQEGDTDAGLKAEEAGIGDTPSLEDEAAGHVTQARM
VSKSKDGTGSDDKKAKGADGKTKIATPRGAAPPGQKGQANATRIPAKTPPAPKTPPSSGEPPKSGDRSG
YSSPGSPGTPGSRSRTPSLPTPPTREPKKVAVVRTPPKSPSSAKSRLQTAPVPMPDLKNVKSKIGSTEN
LKHQPGGGKVQIVYKPVDLSKVTSKCGSLGNIHHKPGGGQVEVKSEKLDFKDRVQSKIGSLDNITHVPG
GGNKKIETHKLTFRENAKAKTDHGAEIVYKSPVVSGDTSPRHLSNVSSTGSIDMVDSPQLATLADEVSA
SLAKQGL (SEQ ID NO: 1)
Tau-B (UniProtKB/Swiss-prot Isoform ID: P10636-4)
381 Amino Acids
MAEPRQEFEVMEDHAGTYGLGDRKDQGGYTMHQDQEGDTDAGLKESPLQTPTEDGSEEPGSETSDAKST
PTAEAEEAGIGDTPSLEDEAAGHVTQARMVSKSKDGTGSDDKKAKGADGKTKIATPRGAAPPGQKGQAN
ATRIPAKTPPAPKTPPSSGEPPKSGDRSGYSSPGSPGTPGSRSRTPSLPTPPTREPKKVAVVRTPPKSP
SSAKSRLQTAPVPMPDLKNVKSKIGSTENLKHQPGGGKVQIVYKPVDLSKVTSKCGSLGNIHHKPGGGQ
VEVKSEKLDFKDRVQSKIGSLDNITHVPGGGNKKIETHKLTFRENAKAKTDHGAEIVYKSPVVSGDTSP
RHLSNVSSTGSIDMVDSPQLATLADEVSASLAKQGL (SEQ ID NO: 2)
Tau-C (UniProtKB/Swiss-prot Isoform ID: P10636-5)
410 Amino Acids
MAEPRQEFEVMEDHAGTYGLGDRKDQGGYTMHQDQEGDTDAGLKESPLQTPTEDGSEEPGSETSDAKST
PTAEDVTAPLVDEGAPGKQAAAQPHTEIPEGTTAEEAGIGDTPSLEDEAAGHVTQARMVSKSKDGTGSD
DKKAKGADGKTKIATPRGAAPPGQKGQANATRIPAKTPPAPKTPPSSGEPPKSGDRSGYSSPGSPGTPG
SRSRTPSLPTPPTREPKKVAVVRTPPKSPSSAKSRLQTAPVPMPDLKNVKSKIGSTENLKHQPGGGKVQ
IVYKPVDLSKVTSKCGSLGNIHHKPGGGQVEVKSEKLDFKDRVQSKIGSLDNITHVPGGGNKKIETHKL
TFRENAKAKTDHGAEIVYKSPVVSGDTSPRHLSNVSSTGSIDMVDSPQLATLADEV
SASLAKQGL (SEQ ID NO: 3)
Tau-D (UniProtKB/Swiss-prot Isoform ID: P10636-6)
383 Amino Acids
MAEPRQEFEVMEDHAGTYGLGDRKDQGGYTMHQDQEGDTDAGLKAEEAGIGDTPSLEDEAAGHVTQARM
VSKSKDGTGSDDKKAKGADGKTKIATPRGAAPPGQKGQANATRIPAKTPPAPKTPPSSGEPPKSGDRSG
YSSPGSPGTPGSRSRTPSLPTPPTREPKKVAVVRTPPKSPSSAKSRLQTAPVPMPDLKNVKSKIGSTEN
LKHQPGGGKVQIINKKLDLSNVQSKCGSKDNIKHVPGGGSVQIVYKPVDLSKVTSKCGSLGNIHHKPGG
GQVEVKSEKLDFKDRVQSKIGSLDNITHVPGGGNKKIETHKLTFRENAKAKTDHGAEIVYKSPVVSGDT
SPRHLSNVSSTGSIDMVDSPQLATLADEVSASLAKQGL (SEQ ID NO: 4)
Tau-E (UniProtKB/Swiss-prot Isoform ID: P10636-7)
412 Amino Acids
MAEPRQEFEVMEDHAGTYGLGDRKDQGGYTMHQDQEGDTDAGLKESPLQTPTEDGSEEPGSETSDAKST
PTAEAEEAGIGDTPSLEDEAAGHVTQARMVSKSKDGTGSDDKKAKGADGKTKIATPRGAAPPGQKGQAN
ATRIPAKTPPAPKTPPSSGEPPKSGDRSGYSSPGSPGTPGSRSRTPSLPTPPTREPKKVAVVRTPPKSP
SSAKSRLQTAPVPMPDLKNVKSKIGSTENLKHQPGGGKVQIINKKLDLSNVQSKCGSKDNIKHVPGGGS
VQIVYKPVDLSKVTSKCGSLGNIHHKPGGGQVEVKSEKLDFKDRVQSKIGSLDNITHVPGGGNKKIETH
KLTFRENAKAKTDHGAEIVYKSPVVSGDTSPRHLSNVSSTGSIDMVDSPQLATLADEV
SASLAKQGL (SEQ ID NO: 5)
Tau-F (UniProtKB/Swiss-prot Isoform ID: P10636-8)
441 Amino Acids
MAEPRQEFEVMEDHAGTYGLGDRKDQGGYTMHQDQEGDTDAGLKESPLQTPTEDGSEEPGSETSDAKST
PTAEDVTAPLVDEGAPGKQAAAQPHTEIPEGTTAEEAGIGDTPSLEDEAAGHVTQARMVSKSKDGTGSD
DKKAKGADGKTKIATPRGAAPPGQKGQANATRIPAKTPPAPKTPPSSGEPPKSGDRSGYSSPGSPGTPG
SRSRTPSLPTPPTREPKKVAVVRTPPKSPSSAKSRLQTAPVPMPDLKNVKSKIGSTENLKHQPGGGKVQ
IINKKLDLSNVQSKCGSKDNIKHVPGGGSVQIVYKPVDLSKVTSKCGSLGNIHHKPGGGQVEVKSEKLD
FKDRVQSKIGSLDNITHVPGGGNKKIETHKLTFRENAKAKTDHGAEIVYKSPVVSGDTSPRHLSNVSST
GSIDMVDSPQLATLADEVSASLAKQGL (SEQ ID NO: 6)
Tau-G (UniProtKB/Swiss-prot Isoform ID: P10636-9)
776 Amino Acids
MAEPRQEFEVMEDHAGTYGLGDRKDQGGYTMHQDQEGDTDAGLKESPLQTPTEDGSEEPGSETSDAKST
PTAEDVTAPLVDEGAPGKQAAAQPHTEIPEGTTAEEAGIGDTPSLEDEAAGHVTQEPESGKVVQEGFLR
EPGPPGLSHQLMSGMPGAPLLPEGPREATRQPSGTGPEDTEGGRHAPELLKHQLLGDLHQEGPPLKGAG
GKERPGSKEEVDEDRDVDESSPQDSPPSKASPAQDGRPPQTAAREATSIPGFPAEGAIPLPVDFLSKVS
TEIPASEPDGPSVGRAKGQDAPLEFTFHVEITPNVQKEQAHSEEHLGRAAFPGAPGEGPEARGPSLGED
TKEADLPEPSEKQPAAAPRGKPVSRVPQLKARMVSKSKDGTGSDDKKAKTSTRSSAKTLKNRPCLSPKL
PTPGSSDPLIQPSSPAVCPEPPSSPKHVSSVTSRTGSSGAKEMKLKGADGKTKIATPRGAAPPGQKGQA
NATRIPAKTPPAPKTPPSSATKQVQRRPPPAGPRSERGEPPKSGDRSGYSSPGSPGTPGSRSRTPSLPT
PPTREPKKVAVVRTPPKSPSSAKSRLQTAPVPMPDLKNVKSKIGSTENLKHQPGGGKVQIINKKLDLSN
VQSKCGSKDNIKHVPGGGSVQIVYKPVDLSKVTSKCGSLGNIHHKPGGGQVEVKSEKLDFKDRVQSKIG
SLDNITHVPGGGNKKIETHKLTFRENAKAKTDHGAEIVYKSPVVSGDTSPRHLSNVSSTGSIDMVDSPQ
LATLADEVSASLAKQGL (SEQ ID NO: 7)
PNS-tau (UniProtKB/Swiss-prot Isoform ID: P10636-1)
758 Amino Acids
MAEPRQEFEVMEDHAGTYGLGDRKDQGGYTMHQDQEGDTDAGLKESPLQTPTEDGSEEPGSETSDAKST
PTAEDVTAPLVDEGAPGKQAAAQPHTEIPEGTTAEEAGIGDTPSLEDEAAGHVTQEPESGKVVQEGFLR
EPGPPGLSHQLMSGMPGAPLLPEGPREATRQPSGTGPEDTEGGRHAPELLKHQLLGDLHQEGPPLKGAG
GKERPGSKEEVDEDRDVDESSPQDSPPSKASPAQDGRPPQTAAREATSIPGFPAEGAIPLPVDFLSKVS
TEIPASEPDGPSVGRAKGQDAPLEFTFHVEITPNVQKEQAHSEEHLGRAAFPGAPGEGPEARGPSLGED
TKEADLPEPSEKQPAAAPRGKPVSRVPQLKARMVSKSKDGTGSDDKKAKTSTRSSAKTLKNRPCLSPKL
PTPGSSDPLIQPSSPAVCPEPPSSPKHVSSVTSRTGSSGAKEMKLKGADGKTKIATPRGAAPPGQKGQA
NATRIPAKTPPAPKTPPSSGEPPKSGDRSGYSSPGSPGTPGSRSRTPSLPTPPTREPKKVAVVRTPPKS
PSSAKSRLQTAPVPMPDLKNVKSKIGSTENLKHQPGGGKVQIINKKLDLSNVQSKCGSKDNIKHVPGGG
SVQIVYKPVDLSKVTSKCGSLGNIHHKPGGGQVEVKSEKLDFKDRVQSKIGSLDNITHVPGGGNKKIET
HKLTFRENAKAKTDHGAEIVYKSPVVSGDTSPRHLSNVSSTGSIDMVDSPQLATLADEV
SASLAKQGL (SEQ ID NO: 8)
Fetal-tau (UniProtKB/Swiss-prot Isoform ID: P10636-2)
316 Amino Acids
MLRALQQRKREAGIGDTPSLEDEAAGHVTQARMVSKSKDGTGSDDKKAKGADGKTKIATPRGAAPPGQK
GQANATRIPAKTPPAPKTPPSSGEPPKSGDRSGYSSPGSPGTPGSRSRTPSLPTPPTREPKKVAVVRTP
PKSPSSAKSRLQTAPVPMPDLKNVKSKIGSTENLKHQPGGGKVQIVYKPVDLSKVTSKCGSLGNIHHKP
GGGQVEVKSEKLDFKDRVQSKIGSLDNITHVPGGGNKKIETHKLTFRENAKAKTDHGAEIVYKSPVVSG
DTSPRHLSNVSSTGSIDMVDSPQLATLADEVSASLAKQGL (SEQ ID NO: 9)
cyclin-dependent kinase 5, regulatory subunit 1
(GenBank Accession No. ACCESSION NP_003876)
307 Amino Acids
MGTVLSLSPSYRKATLFEDGAATVGHYTAVQNSKNAKDKNLKRHSIISVLPWKRIVAVSAKKKNSKKVQ
PNSSYQNNITHLNNENLKKSLSCANLSTFAQPPPAQPPAPPASQLSGSQTGGSSSVKKAPHPAVTSAGT
PKRVIVQASTSELLRCLGEFLCRRCYRLKHLSPTDPVLWLRSVDRSLLLQGWQDQGFITPANVVFLYML
CRDVISSEVGSDHELQAVLLTCLYLSYSYMGNEISYPLKPFLVESCKEAFWDRCLSVINLMSSKMLQIN
ADPHYFTQVFSDLKNESGQEDKKRLLLGLDR (SEQ ID NO: 10)
See, e.g. UniProtKB/Swiss-prot entry P10636 and associated disclosure including that relating to Isoform IDs 1-9 respectively (http://expasy.org/uniprot/P10636).

While specific embodiments of the present invention have been illustrated and described, numerous modifications come to mind without significantly departing from the spirit of the invention and the scope of protection is only limited by the scope of the accompanying claims. All publications listed in the specification are hereby incorporated by reference. Various elements, methods and materials in this art are disclosed for example in: Broadhurst et al. (2000), J. Agric. Food Chem., 48. 1219-1221; Imparl-Radosevich et al. (1998) Hormone Research 50, 177-182; Jarvill-Taylor et al. (2001), Journal of the American College of Nutrition, 20, 327-336; Anderson et al. (2004), Journal of Agricultural and Food Chemistry 52, 65-70; Kahn et al. (2003), Diabetes Care, 26, 3215-3218; Arvanitakis et al., (2005) Arch. Neurol., 61.661-6; Bargjorn et al. (2002), Biochemistry 41, 11120-11126; Schubert et al. (2004), Proc. Nat'l. Acad. Sci (USA), 101,3100-3105; Pickhardt (2005), Curr. Alzheimer Res., April 2, 219-26; Taniguchi et al. (2005), J. of Biol. Chem., 280, 7614-7623; Pickhardt et al. (2005), J. Biol. Chem.,280, 3628-35; Piulcher, Lancet Neuorol., 5, 388-9; U.S Pat. No. 6,200,259; U.S. Pat. No. 7,335,505; Lee et al., Annu Rev Neurosci 24, 1121-59 (2001); Lee et al., Neuron 24, 507-10 (1999); Brandt et al., Biochim Biophys Acta 1739, 331-54 (2005); Avila et al., Physiol Rev 84, 361-84 (2004); Imahori et al., Neurobiol Aging 19, S93-8 (1998); Arvanitakis et al., Arch Neurol 61, 661-6 (2004); Biessels et al., Biochem Soc Trans 33, 1041-4 (2005); Carro et al., Eur J Pharmacol 490, 127-33 (2004); Gasparini et al., Trends Pharmacol Sci 23, 288-93 (2002); Grossman et al., CNS Spectr 8, 815-23 (2003); Haan et al., Nat Clin Pract Neurol 2, 159-66 (2006); Ho et al., Faseb J 18, 902-4 (2004); Rivera et al., J Alzheimer's Dis 8, 247-68 (2005); Schnaider Beeri et al., Neurology 63, 1902-7 (2004); Sun et al., Drugs Today (Barc) 42, 481-9 (2006); Xu et al., Neurology 63, 1181-6 (2004); Watson et al., CNS Drugs 17, 27-45 (2003); Leibson et al., Diabetologia 39, 1392-7 (1996); de la Monte et al., J Alzheimers Dis 7, 45-61 (2005); Hong et al., J Biol Chem 272, 19547-53 (1997); Schubert et al., Proc Nail Acad Sci USA 101, 3100-5 (2004); Roses et al., Alzheimer's and Dementia 2, 59-70 (2006); Khan et al., Biol Trace Elem Res 24, 183-8 (1990); Preuss et al., J Am Coll Nutr 25, 144-50 (2006); Mang et al., Eur J Clin Invest 36, 340-4 (2006); Verspohl et al., Phytother Res 19, 203-6 (2005); Kannappan et al., Singapore Med J 47, 858-63 (2006); Wroblewski, et al., Eur J Biochem 268, 4384-97 (2001); Baxter et al., Biochemistry 36, 5566-77 (1997); de Freitas et al., J Agric Food Chem 49, 940-5 (2001); Wischik et al., Proc Natl Acad Sci USA 93, 11213-8 (1996); Masuda et al., Biochem., 45, 6085-94 (2006); Ferreira et al., Nat Prod Rep 19, 517-41 (2002); Dixon et al., New Phytol 165, 9-28 (2005); von Bergen et al., Proc Natl Acad Sci USA 97, 5129-34 (2000); Khlistunova et al., J Biol Chem 281, 1205-14 (2006); Sato, S. et al. J Biol Chem 277, 42060-5 (2002); Ferrari et al., J Biol Chem 278, 40162-8 (2003); Rapoport et al., Proc Natl Acad Sci USA 99, 6364-9 (2002); Liu, T. et al. J Neurochem 88, 554-63 (2004); Nishimura et al., Cell 116, 671-82 (2004); Mielke et al., J Neurochem 93, 1568-78 (2005); and Andorfer et al., J Neurochem 86, 582-90 (2003).

What is claimed:

1. A method of binding a mammalian tau polypeptide with an isolated proanthocyanidin compris:
combining the tau polypeptide with an isolated proanthocyanidin composition; and
allowing an isolated proanthocyanidin in the composition to bind the tau polypeptide so that the tau polypeptide is bound by the isolated proanthocyanidin.

2. The method of claim 1, wherein the isolated proanthocyanidin bound to the tau polypeptide is used to observe the presence of:
(a) tau polypeptides in a biological sample; or
(b) an aggregation of tau polypeptides in a biological sample.

3. The method of claim 2, wherein the method further comprises using observations of the presence of an aggregation of tau polypeptides to diagnose a tauopathy.

4. The method of claim 1, wherein the isolated proanthocyanidin composition is derived from Cinnamomum loureirii, Cinnamomum cassia or Cinnamomum zyelanicum.

5. The method of claim 1, wherein the isolated proanthocyanidin composition comprises at least one of the following compounds:
(a) an A-linked proanthocyanidin dimer, trimer, tetramer, or pentamer;
(b) a proanthocyanidin B2;
(c) a cinnamaldehyde;
(d) an oxidized catechin; or
(e) an oxidized epicatechin.

6. The method of claim 5, wherein the proanthocyanidin composition comprises all of the compounds (a)-(e).

7. The method of claim 1, wherein the binding of the tau polypeptide by the isolated proanthocyanidin perturbs the ability of the tau polypeptide to form an aggregation of tau polypeptides.

8. The method of claim 7, wherein the binding of the tau polypeptide by the isolated proanthocyanidin perturbs the ability of the tau polypeptide to form an aggregation of tau polypeptides in a tauopathy.

9. The method of claim 1, wherein the tau polypeptide comprises tau-A (SEQ ID NO: 1), tau-B (SEQ ID NO: 2), tau-C (SEQ ID NO: 3), tau-D (SEQ ID NO: 4), tau-E (SEQ ID NO: 5), tau-F (SEQ ID NO: 6) or tau-G (SEQ ID NO: 7) or a proteolytically processed fragment thereof.

10. A method determining if a proanthocyanidin compound binds to a tau polypeptide comprising:
combining the tau polypeptide with a proanthocyanidin compound;
testing the combination of the tau polypeptide and the proanthocyanidin compound to determine if the proanthocyanidin compound binds the tau polypeptide.

11. The method of claim 10, wherein the proanthocyanidin compound is coupled to a detectable marker.

12. A method of determining if an isolated proanthocyanidin compound perturbs aggregation of tau polypeptides comprising:
observing an ability of tau polypeptides to aggregate in an absence of the isolated proanthocyanidin compound; and
combining tau polypeptides with the isolated proanthocyanidin compound and observing the ability of tau polypeptides to aggregate in a presence of the isolated proanthocyanidin compound;
wherein a decrease in tau aggregation observed in the presence of the isolated proanthocyanidin compound as compared to the amount of tau aggregation observed in the absence of the isolated proanthocyanidin compound identifies the isolated proanthocyanidin compound as perturbing the ability of tau polypeptides to aggregate.

13. A process for preparing an isolated proanthocyanidin composition comprising:
(a) extracting a sample of Cinnamomum loureirii, Cinnamomum cassia or Cinnamomum zyelanicum with an aqueous solution;
(b) agitating the extract of (a) for at least one minute at a temperature between 40-90° C.;
(c) centrifuging the agitated extract of (b) so as to produce a first pellet and a first supernatant;
(d) incubating the first supernatant of (c) at 0-4° C. for at least one minute;
(e) centrifuging the incubated supernatant of (d) so as to produce a second pellet and a second supernatant; and
(f) filtering the second supernatant, wherein the resultant filtered second supernatant comprises an aqueous solution of the isolated proanthocyanidin composition.

14. The process of claim 13, further comprising lyophilizing the aqueous solution of the isolated proanthocyanidin composition so as to form a solid isolated proanthocyanidin composition.

15. The process of claim 14, further comprising adding an aqueous solution to the solid isolated proanthocyanidin composition so as to form an aqueous solution of the isolated proanthocyanidin composition.

16. An isolated proanthocyanidin composition product produced by the process of claim 13.

17. An isolated proanthocyanidin compound derived from Cinnamomum loureirii, Cinnamomum cassia or Cinnamomum zyelanicum and characterized by at least one of the following properties:
an ability to bind tau polypeptides;
an ability to reduce the ability of tau polypeptides to form an aggregation as observed by a thioflavin T assays;
an ability to reduce the number and length of tau filament formation as measured by electron microscopy studies;
exhibiting a decrease in the absorbance spectra upon combination with tau polypeptides; or
comprising: an B-linked proanthocyanidin dimer having a mass spectroscopy peak of 617 m/z; an A-linked proanthocyanidin trimer having a mass spectroscopy peak of 905 m/z; an A-linked proanthocyanidin tetramer a mass spectroscopy peak of 1193 m/z; or an A-linked proanthocyanidin pentamer having a mass spectroscopy peak of 1481 m/z; a proanthocyanidin B2; a cinnamaldehyde; an oxidized catechin; or an oxidized epicatechin.

18. A method of treating a mammal having a neurological condition or disorder characterized by an aggregation of tau polypeptides, comprising administering to the mammal an effective amount of an isolated proanthocyanidin composition selected for its ability to perturb aggregation of tau polypeptides.

19. The method of claim 18, wherein the isolated proanthocyanidin composition comprises at least one of the following compounds:
(a) an A-linked proanthocyanidin dimer, trimer, tetramer, or pentamer;
(b) a proanthocyanidin B2;
(c) a cinnamaldehyde;
(d) an oxidized catechin; or
(e) an oxidized epicatechin.

20. The method of claim 18, wherein the neurological condition or disorder is Alzheimer's disease.
